(19) United States
(12) Patent Application Publication
(10) Pub. No.: US 2026/0263473 A1
(43) Pub. Date: Sep. 10, 2026

(54) PRODUCTS OF MANUFACTURE AND THERAPEUTIC COMPOSITIONS FOR TREATING, AMELIORATING OR PREVENTING VIRAL INFECTIONS AND METHODS FOR MAKING AND USING THEM

(71) Applicant: Topelia Australia Ltd., Five Dock (AU)

(72) Inventor: Thomas Julius Borody, Five Dock (AU)

(21) Appl. No.: 19/165,293

(22) PCT Filed: Mar. 15, 2024
(86) PCT No.: PCT/AU2024/050228

§ 371 Date: Sep. 15, 2025

Related U.S. Application Data
(60) Provisional application No. 63/452,399, filed on Mar. 15, 2023.

(51) Int. Cl. A61K 31/53; A61K 9/70; A61K 31/155; A61K 31/65; A61K 31/7048; A61K 31/7052; A61K 33/30; A61K 45/06; A61P 31/14

(52) U.S. Cl. CPC A61K 31/53; A61K 9/7092; A61K 31/155; A61K 31/65; A61K 31/7048; A61K 31/7052; A61K 33/30; A61K 45/06; A61P 31/14

(57) ABSTRACT

In alternative embodiments, provided are pharmaceutical compositions and therapeutic combinations of drugs, including products of manufacture and kits, for treating, preventing or ameliorating (for example, decreasing the symptoms of, or decreasing the mortality of) a viral infection, for example, a coronavirus infection such as a COVID-19 or variant thereof, and methods for making and using same. In alternative embodiments, provided are products of manufacture and kits for delivering pharmaceutical compositions and therapeutic combinations of drugs as provided herein, for example provided are transdermal delivery devices such as patches that can have multiple compartments for delivering transdermally different drugs at the same time.

This application claims the benefit of priority to U.S. Provisional Application No. (U.S. Ser. No.) 63/452,399, filed on 15 Mar. 2023, the entire disclosure of which is incorporated herein by cross-reference.

### FIELD

This invention generally relates to infectious diseases and pharmaceuticals. In alternative embodiments, provided are pharmaceutical compositions and therapeutic combinations of drugs, including products of manufacture and kits, for treating, preventing or ameliorating (for example, decreasing the symptoms of, or decreasing the mortality of) a viral infection, for example, a coronavirus infection such as a COVID-19 or variant thereof, and methods for making and using same. In alternative embodiments, provided are products of manufacture and kits for delivering pharmaceutical compositions and therapeutic combinations of drugs as provided herein.

### BACKGROUND

There have been a number of active single agents that inhibit the growth of Coronavirus. However, when used alone, they do not eradicate the infection quickly enough and many patients still become ill with a cytokine storm and may be admitted to intensive care, and a small percentage will die. Hence, there is a great need to find ways to adequately inhibit the multiplication of the virus intracellularly.

Most of the products that have been included in such therapies had to be used early before the commencement of massive cytokine release, and thus were poorly acting because they were either monotherapies (such as molnupiravir, or LAGEVRIO™) or dual therapies such as combination use of nirmatrelvir and ritonavir as with PAXLOVID™).

### SUMMARY OF INVENTION

In alternative embodiments, provided are pharmaceutical compositions or therapeutic combinations of drugs, comprising:
(a) (i) metformin (or GLUCOPHAGE™) and/or
   (ii) a 3C-like (3CLpro) protease (also known as main protease (Mpro), also known as C30 endopeptidase or 3-chymotrypsin-like protease) inhibitor, optionally ensitrelvir (or XOCOVA™), or ensitrelvir fumaric acid,
wherein optionally the metformin is formulated or dosaged at between about 100 mg to 4000 mg per unit dosage (optionally, per tablet); or the metformin is formulated or dosage at between about 0.1 ml to 25 ml liquid metformin/day; and,
wherein optionally the ensitrelvir or ensitrelvir fumaric acid is formulated or dosaged at between about 100 mg to 500 mg per unit dosage, or at about 100 mg, 125 mg or 150 mg per unit dosage (optionally, per tablet); and
(b) two of any of the following drugs or drug combinations if only metformin or the 3CLpro inhibitor are included, or one of (or at least one of) any of the following drugs or drug combinations if both metformin and 3CLpro are included:
(i) an avermectin class drug, or ivermectin (or STROMECTOL™);
(ii) an avermectin class drug, or ivermectin and an antibiotic (optionally doxycycline or azithromycin);
(iii) an avermectin class drug, or ivermectin; an antibiotic (optionally doxycycline or azithromycin), and zinc, and optionally also vitamin D or cholecalciferol, optionally with or without vitamin C;
(iv) an antibiotic;
(v) ivermectin; and, an antibiotic (optionally doxycycline or azithromycin), at least one vitamin and zinc;
(vi) opaganib or YELIVA™;
(vii) opaganib and: an avermectin class drug, or ivermectin; an antibiotic (optionally doxycycline or azithromycin), and zinc, and optionally also vitamin D or cholecalciferol;
(viii) plitidepsin (also known as dehydrodidemnin B), or APLIDIN™;
(ix) plitidepsin and: an avermectin class drug, or ivermectin; an antibiotic (optionally doxycycline or azithromycin), and zinc, and optionally also vitamin D or cholecalciferol, and optionally also vitamin D or cholecalciferol;
(x) lopinavir, ritonavir (or NORVIR™) and/or oseltamivir,
(xi) lopinavir, ritonavir and/or oseltamivir, and: an avermectin class drug, or ivermectin; an antibiotic (optionally doxycycline or azithromycin), and zinc, and optionally also vitamin D or cholecalciferol;
(xii) nirmatrelvir and ritonavir (or PAXLOVID™),
(xiii) nirmatrelvir and ritonavir (or PAXLOVID™), and: an avermectin class drug, or ivermectin; an antibiotic (optionally doxycycline or azithromycin), and zinc, and optionally also vitamin D or cholecalciferol;
(xiv) chloroquine (optionally, ARALEN™), chloroquine phosphate, chloroquine diphosphate, hydroxychloroquine (optionally, PLAQUENIL™);
(xv) chloroquine, chloroquine phosphate, chloroquine diphosphate, hydroxychloroquine; and: an avermectin class drug, or ivermectin; an antibiotic (optionally doxycycline or azithromycin), and zinc, and optionally also vitamin D or cholecalciferol;
(xvi) abacavir, acyclovir optionally, (ACICLOVIR™), adefovir, amantadine, ampligen, amprenavir (optionally, AGENERASE™), aprepitant, arbidol, atazanavir, atripla, balavir, baloxavir marboxil (XOFLUZA™), bepotastine, bevirimat, bictegravir, biktarvy, brilacidin, cidofovir, caspofungin, lamivudine and zidovudine (optionally, COMBVIR™), cobicstat, colisitin, cocaine, delavirdine, descovy, didanosine, docosanol, dolutegravir, ecoliever, edoxudine, elvitegravir, enfuvirtide, entecavir, epirubicin, epoprostenol, etravirine, famciclovir, fomivirsen, fosamprenavi, foscarnet, fosfonet, galidesivir, ibacitabine, icatibant, idoxuridine, ifenprodil, imiquimod, imunovir, indinavir, inosine, an interferon (optionally interferon type I, interferon type II and/or interferon type III), lamivudine, loviride, ledipasvir, leronlimab, maraviroc, methisazone, moroxydine, nexavir, norvir, a nucleoside analogue (optionally brincidofovir, didanosine, favipiravir (also known as T-705, avigan, or favilavir, Toyama Chemical, Fujifilm, Japan), vidarabine, galidesivir (optionally, BCX4430, IMMUCILLIN-A™), cytarabine, gemcitabine, lamivudine, zalcitabine, abacavir, acyclovir, entecavir, stavudine, telbivudine, zidovudine, idoxuridine and/or trifluridine or any combination thereof), oseltamivir (or TAMIFLU™), peginterferon alfa-2a, penciclovir, peramivir (optionally, RAPIVAB™), perfenazine, pleconaril, plurifloxacin, podophyllotoxin, pyramidine, raltegravir, rifampicin, rilpivirine, rimantadine, ritonavir, saquinavir, sofosbuvir, stavudine, telaprevir, tegobuv, tipranavir, trifluridine, trizivir, tromantadine, truvada, valaciclovir (optionally, VALTREX™), valganciclovir, valrubicin, vapreotide, vicriviroc, vidarabine, viramidine, velpatasvir, vivecon, zalcitabine, zanamivir (optionally, RELENZA™), zidovudine, or any combination thereof;
(xvii) any one or more drug or drugs in (xiv); and: an avermectin class drug, or ivermectin; an antibiotic (optionally doxycycline or azithromycin), and zinc, and optionally also vitamin D or cholecalciferol;
(xviii) molnupiravir, emtricitabine (or EMTRIVA™), efavirenz (or SUSTIVA™) or tenofovir; or molnupiravir with emtricitabine and tenofovir (or ATRIPLA™);
(xix) molnupiravir, emtricitabine, efavirenz or tenofovir, and: an avermectin class drug, or ivermectin; an antibiotic (optionally doxycycline or azithromycin), and zinc, and optionally also vitamin D or cholecalciferol;
(xx) zanamivir (or RELENZA™);
(xxi) zanamivir, and: an avermectin class drug, or ivermectin; an antibiotic (optionally doxycycline or azithromycin), and zinc, and optionally also vitamin D or cholecalciferol;
(xxii) efavirenz (optionally SUSTIVA™);
(xxiii) efavirenz, and: an avermectin class drug, or ivermectin; an antibiotic (optionally doxycycline or azithromycin), and zinc, and optionally also vitamin D or cholecalciferol;
(xxiv) nelfinavir (VIRACEPT™) or oseltamivir (or TAMIFLU™),
(xxv) nelfinavir (VIRACEPT™) or oseltamivir (or TAMIFLU™), and: an avermectin class drug, or ivermectin; an antibiotic (optionally doxycycline or azithromycin), and zinc, and optionally also vitamin D or cholecalciferol;
(xxvi) a thiazolide class drug, optionally nitazoxanide (optionally ALINIA™, or NIZONIDE™) or tizoxanide (or 2-Hydroxy-N-(5-nitro-2-thiazolyl)benzamide);
(xxvii) a thiazolide class drug, optionally nitazoxanide, and: an avermectin class drug, or ivermectin; an antibiotic (optionally doxycycline or azithromycin), and zinc, and optionally also vitamin D or cholecalciferol;
(xxviii) remdesivir (optionally, GS-5734™, Gilead Sciences, or VEKLURY™);
(xxix) molnupiravir (or LAGEVRIO™);
(xxx) molnupiravir, and: an avermectin class drug, or ivermectin; an antibiotic (optionally doxycycline or azithromycin), and zinc, and optionally also vitamin D or cholecalciferol;
(xxxi) nevirapine (or VIRAMUNE™);
(xxxii) nevirapine (or VIRAMUNE™), and: an avermectin class drug, or ivermectin; an antibiotic (optionally doxycycline or azithromycin), and zinc, and optionally also vitamin D or cholecalciferol;
(xxxiii) tenofovir alafenamide, tenofovir disoproxil or tenofovir;
(xxxiv) tenofovir alafenamide, tenofovir disoproxil or tenofovir, and: an avermectin class drug, or ivermectin; an antibiotic (optionally doxycycline or azithromycin), and zinc, and optionally also vitamin D or cholecalciferol;
(xxxv) salamectin (or SELEHOLD™, REVOLT™, SELARID™ or SENERGY™);
(xxxvi) salamectin, and: an avermectin class drug, or ivermectin; an antibiotic (optionally doxycycline or azithromycin), and zinc, and optionally also vitamin D or cholecalciferol;
(xxxvii) an inhibitor of an S-phase kinase-associated protein 2 (SKP2), or dioscin, or niclosamide, or NICLOCIDE™, FENASAL™, or PHENASAL™,
(xxxviii) an inhibitor of an S-phase kinase-associated protein 2 (SKP2), or dioscin, or niclosamide, and: an avermectin class drug, or ivermectin; an antibiotic (optionally doxycycline or azithromycin), and zinc, and optionally also vitamin D or cholecalciferol;
(xxxix) ribavirin or tribavirin (or COPEGUS™, REBETOL™, or VIRAZOLE™);
(xxxx) ribavirin or tribavirin, and: an avermectin class drug, or ivermectin; an antibiotic (optionally doxycycline or azithromycin), and zinc, and optionally also vitamin D or cholecalciferol;
(xxxxi) a viral, or a coronavirus or a COVID-19, protease inhibitor, optionally ASC09 (CAS registry no. 1000287-05-7) (Janssen Research and Development, LLC), ritonavir or ASC 09 and ritonavir, or a JAK1/2 inhibitor (optionally baricitinib), optionally compound 11r (University of Lubeck, Germany, see optionally, Zhang et al J. Med Chem 2020, Feb. 11, 2020), or darunavir (or PREZISTA™), cobicistat (or TYBOST™) or darunavir and cobicistat;
(xxxxii) a viral, or a coronavirus or a COVID-19, protease inhibitor, ritonavir, a JAK1/2 inhibitor (optionally baricitinib), or darunavir (or PREZISTA™) or cobicistat, and: an avermectin class drug, or ivermectin; an antibiotic (optionally doxycycline or azithromycin), and zinc, and optionally also vitamin D or cholecalciferol;
(xxxxiii) an mucolytic therapy or drug, optionally acetylcysteine, ambroxol, bromhexine, carbocisteine, erdosteine, mecysteine or dornase alfa, or an expectorant, optionally guaifenesin;
(xxxxiv) an mucolytic therapy or drug, and: an avermectin class drug, or ivermectin; an antibiotic (optionally doxycycline or azithromycin), and zinc, and optionally also vitamin D or cholecalciferol;
(xxxxv) an angiotensin-converting enzyme 2 (ACE2) inhibitor, optionally captopril, enalapril, lisinopril, benazepril, fosinopril, quinapril, ramipril, perindopril, moexipril or trandolapril;
(xxxxvi) an angiotensin-converting enzyme 2 (ACE2) inhibitor, and: an avermectin class drug, or ivermectin; an antibiotic (optionally doxycycline or azithromycin), and zinc, and optionally also vitamin D or cholecalciferol;
(xxxxvii) an anti-vascular endothelial growth factor (VEGF) (optionally VEGF-A) drug or antibody, optionally bevacizumab (or AVASTIN™);
(xxxxviii) an anti-vascular endothelial growth factor (VEGF) drug or antibody or bevacizumab and: an avermectin class drug, or ivermectin; an antibiotic (optionally doxycycline or azithromycin), and zinc, and optionally also vitamin D or cholecalciferol;
(xxxxix) a protease inhibitor, optionally danoprevir, optionally a serine protease inhibitor, optionally camostat (or FOIPAN™) or narlaprevir (optionally ARLANSA™)
(xxxxx) a protease inhibitor, danoprevir, a serine protease inhibitor, camostat or narlaprevir, and: an avermectin class drug, or ivermectin; an antibiotic (optionally doxycycline or azithromycin), and zinc, and optionally also vitamin D or cholecalciferol;
(xxxxxi) anti-PD-1 checkpoint inhibitor, optionally camrelizumab, or a PD-1 inhibitor (or cemiplimab, dostarlimab, pembrolizumab or nivolumab) or a PD-L1 inhibitor (or atezolizumab, avelumab, cosibelimab or durvalumab);
(xxxxxii) anti-PD-1 checkpoint inhibitor, or camrelizumab, or a PD-1 inhibitor or a PD-L1 inhibitor, and: an avermectin class drug, or ivermectin; an antibiotic (optionally doxycycline or azithromycin), and zinc, and optionally also vitamin D or cholecalciferol;
(xxxxxiii) thalidomide, or thalidomide and glucocorticoid (optionally low-dose glucocorticoid), or and thalidomide and celecoxib;
(xxxxxiv) thalidomide, or thalidomide and glucocorticoid (optionally low-dose glucocorticoid), and: an avermectin class drug, or ivermectin; an antibiotic (optionally doxycycline or azithromycin), and zinc, and optionally also vitamin D or cholecalciferol;
(xxxxxv) a compound or antibody capable of binding complement factor C5 and blocking membrane attack complex formation, or eculizumab;
(xxxxxvi) a compound or antibody capable of binding complement factor C5 and blocking membrane attack complex formation, or eculizumab, and: an avermectin class drug, or ivermectin; an antibiotic (optionally doxycycline or azithromycin), and zinc, and optionally also vitamin D or cholecalciferol;
(xxxxxvii) a cathepsin inhibitor, optionally a cathepsin K, B or L inhibitor, or relacatib or balicatib;
(xxxxxviii) a cathepsin inhibitor, or relacatib or balicatib, and: an avermectin class drug, or ivermectin; an antibiotic (optionally doxycycline or azithromycin), and zinc, and optionally also vitamin D or cholecalciferol;
(xxxxxix) a synthetic nucleoside analog or derivative, or N4-hydroxycytidine, or a prodrug of N4-hydroxycytidine, optionally molnuvpiravir (Merck), or favipiravir (also known as T-705 or AVIGAN™), or favilavir, Toyama Chemical, Fujifilm, Japan, or FABIFLU™ (Glenmark Pharmaceuticals), optionally dosaged at 800 mg bid;
(xxxxxx) a synthetic nucleoside analog or derivative, and: an avermectin class drug, or ivermectin; an antibiotic (optionally doxycycline or azithromycin), and zinc, and optionally also vitamin D or cholecalciferol,
wherein the synthetic nucleoside analog or derivative, or N4-hydroxycytidine, or the prodrug of N4-hydroxycytidine, optionally molnuvpiravir or favipiravir, is given as between about 10 mg to 3 gm per dose, or between about 10 mg to 3 gm per day, or can be dosed either as a single dose or given one, two, three or four times a day, or is administered at 200 to 800 mg twice daily, or 200, 400, 600 or 800 mg twice daily, or at 200 to 800 mg three times a day, or at 200, 400, 600 or 800 mg three times a day, or is administered at 200 to 800 mg three times a day for between about 2 to 15 days, or for about 2, 3, 4, 5, 6, 7, 8, 9, 10, 11 or 12 days, and optionally when combined with other drugs a lower dosage is used, optionally administered at 100 or 200 mg three times a day for between about 5 to 15 days, or for about 7, 8, 9, 10, 11 or 12 days;
(xxxxxxi) an anti-androgen drug, and optionally the anti-androgen drug is bicalutamide, or CASODEX™, or dutasteride (or AVODART™), and optionally the anti-androgen drug comprises a 5α-reductase inhibitor, and optionally the 5α-reductase inhibitor comprises finasteride (or PROSCAR™, PROPECIA™, or FINIDE™);
(xxxxxxii) an anti-androgen drug, and optionally the anti-androgen drug is bicalutamide, or CASODEX™, or dutasteride (or AVODART∩), and: an avermectin class drug, or ivermectin; an antibiotic (optionally doxycycline or azithromycin), and zinc, and optionally also vitamin D or cholecalciferol;
(xxxxxxiii) an anti-malarial drug, wherein optionally the anti-malarial drug comprises mefloquine (or LARIAM™, MEPHAQUIN™, or MEFLIAM™), wherein optionally the mefloquine is formulated for oral administration, optionally in tablet or capsule form, optionally as 200 mg, 250 mg or 300 mg tablets;
(xxxxxxiv) an anti-malarial drug, and: an avermectin class drug, or ivermectin; an antibiotic (optionally doxycycline or azithromycin), and zinc, and optionally also vitamin D or cholecalciferol;
(xxxxxxv) a peroxisome proliferator-activated receptor (PPAR) agonist, wherein optionally the PPAR agonist comprises fenofibrate, or TRICOR™, FENOBRAT™, FENOGLIDE™ or LIPOFEN™, optionally the PPAR agonist comprises a combination of fenofibrate and pravastatin, or PRAVAFENIX™, or the PPAR agonist comprises bezafibrate, or BEZALIP™, or combination of bezafibrate and chenodeoxycholic acid, or HEPACONDA™, or aluminium clofibrate, or alfibrate, or ciprofibrate, or clinofibrate or LIPOCLIN™, or clofibrate or ATROMID-S™, or clofibride, or gemfibrozil or LOPID™, or ronifibrate, or simfibrate or CHOLESOLVIN™, or any combination thereof,
(xxxxxxvi) a peroxisome proliferator-activated receptor (PPAR) agonist, and: an avermectin class drug, or ivermectin; an antibiotic (optionally doxycycline or azithromycin), and zinc, and optionally also vitamin D or cholecalciferol;
(xxxxxxvii) an acetaldehyde dehydrogenase inhibitor, optionally disulfiram, or ANTABUS™, or ANTABUSE™, optionally formulated as an extended, sustained or slow-release disulfiram formulation, optionally the extended, sustained or slow-release disulfiram is formulated as a tablet, a capsule or in an injectable, amphiphilic, absorbable, depot-forming drug delivery system (DDS),
(xxxxxxviii) an acetaldehyde dehydrogenase inhibitor, and: an avermectin class drug, or ivermectin; an antibiotic (optionally doxycycline or azithromycin), and zinc, and optionally also vitamin D or cholecalciferol;
(xxxxxxix) a nicotinic antagonist, a dopamine agonist or a noncompetitive N-Methyl-D-aspartate (NMDA) antagonist, optionally amantadine, or GOCOVRI™, or SYMADINE™, or SYMMETREL™, optionally dosaged at between about 100 to 200 mg per dose, optionally formulated as tablets or capsules,
(xxxxxxx) a nicotinic antagonist, a dopamine agonist or a noncompetitive N-Methyl-D-aspartate (NMDA) antagonist, and: an avermectin class drug, or ivermectin; an antibiotic (optionally doxycycline or azithromycin), and zinc, and optionally also vitamin D or cholecalciferol;
(xxxxxxxi) a mitochondrial sensitizer, optionally proguanil or chlorguanide (or PALUDRINE™), or a malarial cytochrome bel complex inhibitor, optionally atovaquone (or MEPRON™), or a combination of proguanil and atovaquone (or MALARONE™),
and optionally the proguanil, atovaquone or the combination of proguanil and atovaquone are formulated for oral administration, optionally as tablets, optionally the unit dosage of atovaquone is 250 mg, 300 mg, 350 mg, 400 mg, 500 mg or 1 gram, and the unit dosage of proguanil is 100 mg, 250 mg, 300 mg, 350 mg or 400 mg;
(xxxxxxxii) a mitochondrial sensitizer or a malarial cytochrome bel complex inhibitor, and: an avermectin class drug, or ivermectin; an antibiotic (optionally doxycycline or azithromycin), and zinc, and optionally also vitamin D or cholecalciferol;
(xxxxxxxiii) a dendrimer, optionally astodrimer sodium (Starpharma, Melbourne, Australia);
(xxxxxxxiv) a dendrimer, and: an avermectin class drug, or ivermectin; an antibiotic (optionally doxycycline or azithromycin), and zinc, and optionally also vitamin D or cholecalciferol;
(xxxxxxxv) an antihistamine class drug, optionally azelastine, or ASTELIN™, OPTIVAR™, ALLERGODIL™, bepotastine (or TALION™, BEPREVE™), brompheniramine, fexofenadine or ALLEGRA™, pheniramine or AVIL™, or chlorpheniramine;
(xxxxxxxvi) an antihistamine class drug, and: an avermectin class drug, or ivermectin; an antibiotic (optionally doxycycline or azithromycin), and zinc, and optionally also vitamin D or cholecalciferol;
(xxxxxxxvii) a selective serotonin reuptake inhibitor (SSRI) class drug, optionally fluvoxamine, or LUVOX™, FAVERIN™, FLUVOXIN™; a peroxisome proliferator-activated receptor (PPAR) agonist, wherein optionally the PPAR agonist comprises fenofibrate, or TRICOR™, FENOBRAT™, FENOGLIDE™ or LIPOFEN™, optionally the PPAR agonist comprises a combination of fenofibrate and pravastatin, or PRAVAFENIX™, or the PPAR agonist comprises bezafibrate, or BEZALIP™, or combination of bezafibrate and chenodeoxycholic acid, or HEPACONDA™, or aluminium clofibrate, or alfibrate, or ciprofibrate, or clinofibrate or LIPOCLIN™, or clofibrate or ATROMID-S™, or clofibride, or gemfibrozil or LOPID™, or ronifibrate, or simfibrate or CHOLESOLVIN™, or any combination thereof;
(xxxxxxxviii) a selective serotonin reuptake inhibitor (SSRI) class drug, and: an avermectin class drug, or ivermectin; an antibiotic (optionally doxycycline or azithromycin), and zinc, and optionally also vitamin D or cholecalciferol;
(xxxxxxxix) clofazimine, or LAMPENE™, optionally dosaged at about 100 mg per day, or between about 50 mg and 150 mg per day, and/or colchicine (or COLCRYS™, MITIGARE™), optionally dosaged at about 0.1 mg to 5 mg per day;
(xxxxxxxx) clofazimine or colchicine, and: an avermectin class drug, or ivermectin; an antibiotic (optionally doxycycline or azithromycin), and zinc, and optionally also vitamin D or cholecalciferol;
(xxxxxxxxi) a selective estrogen receptor modulator (SERM), or toremifene (or FARESTON™), or clomifene or clomiphene (or CLOMID™, SEROPHENE™);
(xxxxxxxxii) a selective estrogen receptor modulator (SERM), or toremifene or clomifene or clomiphene, and: an avermectin class drug, or ivermectin; an antibiotic (optionally doxycycline or azithromycin), and zinc, and optionally also vitamin D or cholecalciferol;
(xxxxxxxxiii) an alpha-ketoamide (α-ketoamide), wherein optionally the alpha-ketoamide is a structure as described by Zhang et al, J. Med. Chem. 2020, 63, 9, 4562-4578, or Meng et al Chem. Sci. (2019) vol. 10, pg 5156 (optionally the structure KAM-2);
(xxxxxxxxiv) an alpha-ketoamide, and: an avermectin class drug, or ivermectin; an antibiotic (optionally doxycycline or azithromycin), and zinc, and optionally also vitamin D or cholecalciferol;
(xxxxxxxxv) an acylsulfonamide inhibitor of the NS3-4A serine protease, or paritaprevir, or ombitasvir, or paritaprevir and ombitasvir with ritonavir (TECHNIVIE™);
(xxxxxxxxvi) paritaprevir (or VERUPREVIR™), or ombitasvir, or paritaprevir and ombitasvir with ritonavir, and: an avermectin class drug, or ivermectin; an antibiotic (optionally doxycycline or azithromycin), and zinc, and optionally also vitamin D or cholecalciferol;
(xxxxxxxxvii) at least one vitamin, and optionally the at least one vitamin comprises: vitamin B3 (or pyridine-3-carboxylic acid, niacin or nicotinic acid, or vitamin B3 or niacin administered as a slow release form (or NIASPAN FCT™), vitamin D (optionally D2, or ergocalciferol), or Vitamin D3 or cholecalciferol, optionally administered at about 1000 to 4000 ugm/day; vitamin B12, vitamin B6 (or pyridoxine); vitamin K; vitamin A; vitamin E; and/or, vitamin C (optionally administered at 500 mg bid);
(xxxxxxxxviii) at least one vitamin, and: an avermectin class drug, or ivermectin; an antibiotic (optionally doxycycline or azithromycin), and zinc;
(xxxxxxxxix) a compound, drug or formulation that decreases stomach acid production or decreases stomach pH, wherein optionally the compound, drug or formulation comprises famotidine, or PEPCID™, and optionally the famotidine is administered at a dosage of between about 10 to 60 mg per day, or between about 20 to 40 mg per day;
(xxxxxxxxx) famotidine, and: an avermectin class drug, or ivermectin; an antibiotic (optionally doxycycline or azithromycin), and zinc;
(xxxxxxxxxi) an immunosuppressive drug, wherein optionally the immunosuppressive drug comprises tocilizumab or atlizumab, or ACTEMRA™, or ROACTEMRA™, or a calcineurin inhibitor (CNI), wherein the CNI comprises ciclosporin (or cyclosporine or cyclosporin), or NEORAL™, or SANDIMMUNE™, or tacrolimus, or PROTOPIC™, or PROGRAF™.
(xxxxxxxxxii) an immunosuppressive drug or a calcineurin inhibitor, and: an avermectin class drug, or ivermectin; an antibiotic (optionally doxycycline or azithromycin), and zinc;
(xxxxxxxxxiii) an anti-inflammatory therapy or at least one anti-inflammatory therapy drug, wherein optionally the anti-inflammatory therapy or drug comprises: a sphingosine kinase-2 (SK2) selective inhibitor (optionally, opaganib (optionally, YELIVA™), sirolimus, a JAK1/2/TYK2 inhibitor (optionally ruxolitinib), an anti-CD47 mAb (optionally meplazumab), a cyclooxygenase (COX) (optionally, COX2) inhibitor, a glucocorticoid (optionally a synthetic glucocorticoid, hydrocortisone, dexamethasone (or DEXTENZA™, OZURDEX™, or NEOFORDEX™) or cortisol, or CORTEF™), plitidepsin or dehydrodidemnin B, or APLIDIN™; and/or
(xxxxxxxxxiv) an anti-inflammatory therapy or at least one anti-inflammatory therapy drug, and: an avermectin class drug, or ivermectin; an antibiotic (optionally doxycycline or azithromycin), and zinc;
wherein optionally the avermectin class drug comprises: ivermectin (or STROMECTOL™), moxidectin (or CYDECTIN™, EQUEST™, QUEST™), selamectin (or STRONGHOLD™), a milbemycin (optionally milbemectin, milbemycin oxime, moxidectin or nemadectin), doramectin (or DECTOMAX™), eprinomectin or abamectin,
and optionally the avermectin class drug or ivermectin is dosaged for administration at between about 50 to 2000 μgm/kg body weight per day, optionally dosaged at 200 μgm/kg per day, or about 14 mg/day, optionally dosaged at 220, 225, 150, 275 or 300 μgm/kg per day, or optionally dosaged at 12, 14, 16, 18, 20, 22, 24 or 26 mg per day, or optionally dosaged at between about 10 to 30 mg per day,
and optionally the avermectin class drug or ivermectin is dosaged for administration at between about 5 to 480 or between about 3 to 500 mg per day,
and optionally the avermectin class drug (optionally ivermectin) is formulated or dosaged at between about 15 to 150 mg/kg, or is about 18, 24, 30, 35, 40, 35, 50, 55, 60, 65, 70, 75, 80, 85, 90, 95, 100, 110 or 120 or more mg/kg,
and optionally the antibiotic comprises a tetracycline class drug, and optionally the tetracycline class drug comprises tetracycline, doxycycline, chlortetracycline, oxytetracycline, glycylcycline, fluorocycline or demeclocycline, tetracycline or SUMYCIN™; chlortetracycline or AUREOMYCIN™; oxytetracycline; demeclocycline or DECLOMYCIN™ DECLOSTATIN™, LEDERMYCIN™, BIOTERCICLIN™, DEGANOL™, DETECLO™, DETRAVIS™, MECICLIN™, MEXOCINE™, CLORTETRIN™; lymecycline; meclocycline; metacycline; minocycline or MINOCIN™; rolitetracycline; doxycycline, or DORYX™, DOXYHEXA™, DOXYLIN™; tigecycline or TYGACIL™; eravacycline or XERAVA™; sarecycline or SEYSARA™; omadacycline or NUZYRA™; or any combination thereof,
and optionally the therapeutic combination comprises about 25 mg to about 600 mg of the tetracycline class drug, or tetracycline, doxycycline, chlortetracycline, oxytetracycline or demeclocycline,
and optionally the antibiotic comprises a macrolide drug, and optionally the macrolide drug comprises azithromycin, optionally dosaged at between about 50 mg to about 2000 mg per dose or per day (optionally, ZITHROMAX™, or AZITHROCIN™, optionally an oral extended- or delayed-release formulation of azithromycin, or ZMAX™), clarithromycin (optionally, BIAXIN™), erythromycin (optionally, ERYTHROCIN™), or fidaxomicin (optionally, DIFICID™ or DIFICLIR™), troleandomycin (optionally, TEKMISIN™), tylosin (optionally, TYLOCINE™ or TYLAN™), solithromycin (optionally, SOLITHERA™), oleandomycin (or SIGMAMYCINE™), midecamycin, roxithromycin, kitasamycin or turimycin, josamycin, carbomycin or magnamycin, and/or spiramycin,
and optionally the antibiotic comprises azithromycin, and optionally the therapeutic combination comprises between about 50 mg to about 2000 mg azithromycin, or optionally the azithromycin comprises an oral extended-release formulation of azithromycin,
and optionally the antibiotic comprises a nitroimidazole, or a 2-, 4- and/or 5-nitroimidazole, or a 5-nitro nitroimidazole comprising metronidazole, tinidazole, nimorazole, dimetridazole, pretomanid, ornidazole, megazol and/or azanidazole, or a 2-nitromidazoles comprising benznidazole,
and optionally the antibiotic comprises a β-lactam antibiotic such as penicillin class drug or a cephalosporin class drug,
and optionally the cephalosporin class drug comprises cefalexin or cephalexin, cefadroxil or cefadroxyl, cefazolin or cephazolin, cefapirin or cephapirin, cefalotin or cephalothn, cefradine or corcephradine, cefprozil or cefproxil, cefuroxime, ceftolozane, cefonicid or cefaclor,
and optionally the penicillin class drug comprises penicillin V, penicillin G, cloxacillin, dicloxacillin, flucloxacillin, methicillin, nafcillin, oxacillin, ampicillin, amoxicillin, pivampicillin, bacampicillin, metampicillin, talampicillin, hetacillin, carbenicillin, ticarcillin, temocillin, mezlocillin, piperacillin, azlocillin, clavulanic acid, sulbactam or tazobactam,
and optionally the antibiotic comprises a quinolone class antibiotic such as a fluoroquinolone, or comprises ciprofloxacin, garenoxacin, gatifloxacin, gemifloxacin, levofloxacin or moxifloxacin,
and optionally the antibiotic comprises an ansamycin class drug, or rifampicin, also known as rifampin, bedaquiline,
and optionally the antibiotic comprises artemisinin, amodiaquine, proguanil, sulfadoxine, sulfamethoxypyridazine, pyrimethamine, linezolid, quinine, quinidine, cinchonine, cinchonidine (or quinimax), hydroxychloroquine or chloroquine,
and optionally the at least one vitamin comprises vitamin D or cholecalciferol, or vitamin C, or vitamin D or cholecalciferol and vitamin C, and vitamin D or cholecalciferol is dosaged at between about 3,000 to about 100,000 units vitamin D or cholecalciferol, and optionally the therapeutic combination comprises between about 10,000 to about 50,000 units vitamin D or cholecalciferol, and optionally the vitamin comprises vitamin C, and optionally the vitamin C is formulated or administered at a dosage of between about 500 to 5000 units (U) per dose,
and optionally the zinc comprises or is formulated as: a zinc salt, a zinc chelate, a zinc sulphate, a zinc acetate, a zinc gluconate or a zinc picolinate, and optionally the therapeutic combination comprises between about 1 mg to 250 mg zinc, zinc sulphate, zinc acetate, zinc gluconate or zinc picolinate, or zinc oxide nanoparticles, optionally at a dosage of between about 1 mg to 250 mg,
and optionally the pharmaceutical composition or a therapeutic combination further comprises copper, optionally administered or formulated at a dosage of between about 1 to 200 mg per day, wherein optionally the copper is administered or formulated as cupric chloride and administered intravenously formulated at about 0.4 mg/ml;
and optionally the pharmaceutical composition or a therapeutic combination further comprises selenium, optionally administered as selenious acid formulated at about 65.4 mcg/ml (or μ/ml), and optionally the selenium is administered at a dosage of between about 50 to 100 μ/ml, optionally between about 60 to 100 μgm per day is administered to an adult, and only up to 60 μgm per day for pediatric patients.

In alternative embodiments, provided are products of manufacture comprising a pharmaceutical composition or a therapeutic combination of drugs as provided herein,
wherein optionally the product of manufacture comprises a pharmaceutical formulation, or a dressing or a dermal patch, or an implant,
and optionally the pharmaceutical composition or a therapeutic combination is formulated as: gel, a liquid, an aerosol, a spray, a mist, a lotion, a cream, a powder, a tablet, a capsule, a pill, a geltab, an enema, a suppository, a transdermal or intraoral patch, or an injectable formulation, a subcutaneous, an intramuscular (IM) or intravenous (IV) formulation, to make a pharmaceutical formulation.

In alternative embodiments, provided are kits comprising a pharmaceutical composition or a therapeutic combination of drugs as provided herein, or a product of manufacture as provided herein, optionally to deliver both oral and transdermal medications as needed.

In alternative embodiments, provided are uses of a pharmaceutical composition or a therapeutic combination of drugs as provided herein, or a product of manufacture as provided herein, or a kit as provided herein, for treating, preventing or ameliorating (optionally decreasing the symptoms of, or decreasing the mortality of) an infection caused by a virus, optionally a virus in the sub family Ortho-coronavirinae, or a virus in the family Coronaviridae, Arteriviridae, Roniviridae and/or Mesoniviridae, or a virus in the subfamily subfamily Letovirinae and Orthocoronavirinae; or a virus in the order Nidovirales, or an Alphacoronavirus, betacoronavirus, gammacoronavirus or deltacoronavirus, or a severe acute respiratory syndrome coronavirus 2 (SARS-COV-2) virus or variant thereof, and optionally the SARS-COV-2 is an omicron variant, or a B.1.1.529, BA.1, BA.1.1, BA.2, BA.3, BA.4 and BA.5 lineage variant; or optionally the SARS-COV-2 is an alpha, beta, gamma, delta, epsilon, eta, iota, kappa, mu or zeta variant,
and optionally the coronavirus infection is a viral infection caused by COVID-19 or a variant, alternative clade or substrain thereof, or a Middle East respiratory syndrome virus (MERS-COV) infection, and optionally the coronavirus infection is a so-called “long COVID” or Post-COVID Conditions (PCC), or post-acute sequelae of SARS COV-2 infection (PASC),
and optionally the viral infection is caused by: a virus that causes a common cold, an influenza virus (optionally an influenza A, B or C), a hepatitis virus, a rous sarcoma virus (RSV), a Paramyxoviridae or measles virus, a Paramyxovirus or mumps virus, a Herpes simplex virus (HSV), a Cytomegalovirus (CMV), a Rubivirus or rubella virus, an Enterovirus, a viral meningitis, a rhinovirus, a human immunodeficiency virus (HIV), a varicella-zoster or chickenpox virus, an Orthopoxvirus or variola or smallpox virus, an Epstein-Barr virus (EBV), an Adenovirus, a Hantavirus, a Flaviviridae or Dengue virus, a Zika virus, or a chikungunya virus infection.

In alternative embodiments, provided are uses of a pharmaceutical composition or a therapeutic combination of drugs as provided herein, or a product of manufacture as provided herein, or a kit as provided herein, for the manufacture of a medicament for treating, preventing or ameliorating (optionally decreasing the symptoms of, or decreasing the mortality of) an infection caused by a virus, optionally a virus in the sub family Ortho-coronavirinae, or a virus in the family Coronaviridae, Arteriviridae, Roniviridae and/or Mesoniviridae, or a virus in the subfamily subfamily Letovirinae and Orthocoronavirinae; or a virus in the order Nidovirales, or an Alphacoronavirus, betacoronavirus, gammacoronavirus or deltacoronavirus, or a severe acute respiratory syndrome coronavirus 2 (SARS-COV-2) virus or variant thereof, and optionally the SARS-COV-2 is an omicron variant, or a B.1.1.529, BA.1, BA.1.1, BA.2, BA.3, BA.4 and BA.5 lineage variant; or optionally the SARS-COV-2 is an alpha, beta, gamma, delta, epsilon, eta, iota, kappa, mu or zeta variant,
and optionally the coronavirus infection is a viral infection caused by COVID-19 or a variant, alternative clade or substrain thereof, or a Middle East respiratory syndrome virus (MERS-COV) infection, and optionally the coronavirus infection is a so-called “long COVID” or Post-COVID Conditions (PCC), or post-acute sequelae of SARS COV-2 infection (PASC),
   and optionally the viral infection is caused by: a virus that causes a common cold, an influenza virus (optionally an influenza A, B or C), a hepatitis virus, a rous sarcoma virus (RSV), a Paramyxoviridae or measles virus, a Paramyxovirus or mumps virus, a Herpes simplex virus (HSV), a Cytomegalovirus (CMV), a Rubivirus or rubella virus, an Enterovirus, a viral meningitis, a rhinovirus, a human immunodeficiency virus (HIV), a varicella-zoster or chickenpox virus, an Orthopoxvirus or variola or smallpox virus, an Epstein-Barr virus (EBV), an Adenovirus, a Hantavirus, a Flaviviridae or Dengue virus, a Zika virus, or a chikungunya virus infection.

In alternative embodiments, provided are pharmaceutical compositions or a therapeutic combination of drugs as provided herein, or a product of manufacture as provided herein, or a kit as provided herein, for use in treating, preventing or ameliorating (optionally decreasing the symptoms of, or decreasing the mortality of) an infection caused by a virus, optionally a virus in the subfamily Ortho-coronavirinae, or a virus in the family Coronaviridae, Arteriviridae, Roniviridae and/or Mesoniviridae, or a virus in the subfamily subfamily Letovirinae and Orthocoronavirinae; or a virus in the order Nidovirales, or an Alphacoronavirus, betacoronavirus, gammacoronavirus or deltacoronavirus, or a severe acute respiratory syndrome coronavirus 2 (SARS-COV-2) virus or variant thereof, and optionally the SARS-COV-2 is an omicron variant, or a B.1.1.529, BA.1, BA.1.1, BA.2, BA.3, BA.4 and BA.5 lineage variant; or optionally the SARS-COV-2 is an alpha, beta, gamma, delta, epsilon, eta, iota, kappa, mu or zeta variant,
and optionally the coronavirus infection is a viral infection caused by COVID-19 or a variant, alternative clade or substrain thereof, or a Middle East respiratory syndrome virus (MERS-COV) infection,
and optionally the viral infection is caused by: a virus that causes a common cold, an influenza virus (optionally an influenza A, B or C), a hepatitis virus, a rous sarcoma virus (RSV), a Paramyxoviridae or measles virus, a Paramyxovirus or mumps virus, a Herpes simplex virus (HSV), mumps virus (MuV), Human papillomavirus virus (HPV), a Cytomegalovirus (CMV), a Rubivirus or rubella virus, an Enterovirus, a viral meningitis, a vaccinia virus, a rhinovirus, a human immunodeficiency virus (HIV), a varicella-zoster or chickenpox virus, an Orthopoxvirus or variola or smallpox virus, an Epstein-Barr virus (EBV), an Adenovirus, a Hantavirus, a Flaviviridae or Dengue virus, ebola virus, a Zika virus, a pneumonia virus, a norovirus, a rotavirus, a virus of the genus Enterovirus or poliovirus, or a chikungunya virus infection.

In alternative embodiments, provided are methods for treating, preventing or ameliorating (optionally decreasing the symptoms of, or decreasing the mortality of) an infection caused by a virus, optionally a virus in the subfamily Ortho-coronavirinae, or a virus in the family Coronaviridae, Arteriviridae, Roniviridae and/or Mesoniviridae, or a virus in the subfamily subfamily Letovirinae and Orthocoronavirinae; or a virus in the order Nidovirales, or an Alphacoronavirus, betacoronavirus, gammacoronavirus or deltacoronavirus, or a severe acute respiratory syndrome coronavirus 2 (SARS-COV-2) virus or variant thereof, and optionally the SARS-COV-2 is an omicron variant, or a B.1.1.529, BA.1, BA.1.1, BA.2, BA.3, BA.4 and BA.5 lineage variant; or optionally the SARS-COV-2 is an alpha, beta, gamma, delta, epsilon, eta, iota, kappa, mu or zeta variant,
and optionally the coronavirus infection is a viral infection caused by COVID-19 or a variant, alternative clade or substrain thereof, or a Middle East respiratory syndrome virus (MERS-COV) infection,
and optionally the viral infection is caused by: a virus that causes a common cold, an influenza virus (optionally an influenza A, B or C), a hepatitis virus, a rous sarcoma virus (RSV), a Paramyxoviridae or measles virus, a Paramyxovirus or mumps virus, a Herpes simplex virus (HSV), a Cytomegalovirus (CMV), a Rubivirus or rubella virus, an Enterovirus, a viral meningitis, a rhinovirus, a human immunodeficiency virus (HIV), a varicella-zoster or chickenpox virus, an Orthopoxvirus or variola or smallpox virus, an Epstein-Barr virus (EBV), an Adenovirus, a Hantavirus, a Flaviviridae or Dengue virus, a Zika virus, or a chikungunya virus infection,
the method comprising:
administering to an individual in need thereof of a pharmaceutically effective amount of a pharmaceutical composition or a therapeutic combination of drugs of as provided herein, or a product of manufacture as provided herein, or a kit as provided herein.

In alternative embodiments, of methods as provided herein:
the avermectin class drug (optionally ivermectin) is dosage or administered:
(a) (i) in or as a loading dosage comprising an avermectin class drug (optionally ivermectin) in a dosage of:
   (1) about 300 μg/kg to 30 mg/kg (or 30 mg per 2.2 pounds (lb)) or about 18 mg to 1800 mg in a 60 kg (about 132 lb), or a dosage of 50 μg/kg, 75 μg/kg, 100 μg/kg or 500 μg/kg, or a dosage of between about 50 μg/kg and 500 μg/kg, or a loading dose of an avermectin class drug (optionally ivermectin) of between about 300 μg/kg per day to between 30 mg/kg to 60 mg/kg or between about 18 mg to about 1200 mg or 1600 mg to 1800 mg in a 60 kg (about 132 lb) person, or between about 300 μg (mcg) to about 40 to 70 mg/kg, or a dosage of 60 to 120 mg to about 1600 to 1800 mg for an adult per day; or,
   (2) between about 18 to 50 mg, or about 18 mg, 24 mg, 30 mg, 36 mg or 40 mg, or between about 50 mg to 100 mg, or 60 to 120 mg up to about 1600 to 1800 mg for an adult; and
wherein optionally the loading dosage is given once, or daily, or periodically, optionally every 2, 3, 4, 5, 6, 7, 8, 9, 10, 11, or 12 or more days,
(ii) after administration of the loading dosage of (i), administering a maintenance dosage of ivermectin of between about 20 mcg/kg (μ/kg) to 5000 mcg/kg (μ/kg) or between about 200 to 2000 mcg/kg (μ/kg) per dose, where 200 mcg/kg is equivalent to a 12 mg dosage in a 60 kg adult, and 2000 mcg/kg is equivalent to 120 mg per dose, or at about 50 μg/kg, 75 μg/kg or 100 μg/kg,
wherein optionally the maintenance dosage is administered 1, 2, 3, 4, 5, 6, 7, 8, 9, 10, 11, 12, 13 or 14 days, or every 3 weeks or every month or every two months or longer after the first loading dosage,
wherein optionally the maintenance dosage is administered every 1, 2, 3, 4, 5, 6, 7, 8, 9, 10, 11, 12, 13 or 14 days, every 3 weeks, or monthly, over the 4 to 8 weeks, 6 to 10 weeks, 8 to 12 weeks, 10 to 20 weeks, 15 to 30 weeks or 20 to 52 weeks, or more, after the initial or loading dose is given,
wherein optionally: an antibiotic or anti-viral is administered with the loading dosage of the avermectin class drug (optionally ivermectin); zinc (optionally zinc chelate, zinc salt or zinc sulfate) is administered with the loading dosage of the avermectin class drug (optionally ivermectin),
and optionally a drug combination, optionally formulated as one formulation (for example, as a tablet capsule) comprises: ivermectin, doxycycline and zinc chelate, or comprises: ivermectin 12 mg, doxycycline 100 mg and zinc chelate 25 mg, optionally administered once or twice daily; or
(b) a drug, a formulation or a therapeutic combination of drugs comprising an avermectin class drug (optionally ivermectin) at a dosage of:
   (i) about 300 μg/kg to 30 mg/kg (or 30 mg per 2.2 pounds (lb)) or about 18 mg to 1800 mg in a 60 kg (about 132 lb), or a dosage of 50 μg/kg, 75 μg/kg or 100 μg/kg, or at a loading dose of ivermectin of between about 30 μg/kg to 60 mg/kg or between about 18 mg to about 1200 mg or 1600 mg to 1800 mg in a 60 kg (about 132 lb) person, or between about 40 to 70 mg/kg, or a dosage of 60 to 120 mg to about 1600 to 1800 mg, or is dosage at 50 μg/kg, 75 μg/kg, 100 μg/kg or 500 μg/kg, or between about 50 μg/kg and 500 μg/kg, for an adult, or
   (ii) between about 18 to 50 mg, or about 18 mg, 24 mg, 30 mg, 36 mg or 40 mg, or between about 50 mg to 100 mg, or 60 to 120 mg to about 1600 to 1800 mg for daily an adult;
an additional drug or drugs is dosaged or administered with the metformin and/or the 3C-like (3CLpro) protease or ensitrelvir, wherein the additional drug or drugs comprises:
(a) combination of an avermectin class drug (optionally ivermectin) (optionally dosaged at between about 30 to 80 mg per day, or between about 36 to 60 mg per day), clofazimine (optionally dosaged at about 100 mg or 150 mg per day, or between about 50 mg and 200 mg per day) and zinc (optionally a zinc sulphate, acetate, gluconate or picolinate, or zinc oxide nanoparticles, optionally at a dosage of between about 1 mg to 250 mg, or about 50 mg per day), and optionally also including colchicine;
(b) a combination of clofazimine (optionally dosaged at about 100 mg or 150 mg per day, or between about 50 mg and 200 mg per day), fluvoxamine, and zinc (optionally a zinc sulphate, acetate, gluconate or picolinate, or zinc oxide nanoparticles, optionally at a dosage of between about 1 mg to 250 mg, or about 50 mg per day), and optionally also including colchicine; or
(c) a combination of an avermectin class drug (optionally ivermectin) (optionally dosaged at between about 10 to 100 mg per day or between about 12 to 80 mg per day, or between about 36 to 60 mg per day), clofazimine (optionally dosaged at about 100 mg or 150 mg per day, or between about 50 mg and 200 mg per day), fluvoxamine and at least one vitamin, wherein optionally the at least one vitamin comprises: vitamin B3 (or pyridine-3-carboxylic acid, niacin or nicotinic acid, or vitamin B3 or niacin administered as a slow release form (or NIASPAN FCT™), vitamin D (optionally D2, or ergocalciferol), or Vitamin D3 or cholecalciferol, optionally administered at about 1000 to 4000 μgm/day; vitamin B12, vitamin B6 (or pyridoxine); vitamin K; vitamin A; vitamin E; and/or, vitamin C (optionally administered at 500 mg bid), and optionally further comprising zinc (optionally a zinc sulphate, acetate, gluconate or picolinate, or zinc oxide nanoparticles, optionally at a dosage of between about 1 mg to 250 mg), and optionally also including colchicine; hydrocortisone or cortisol (optionally CORTEF™, SOLUCORTEF™), optionally hydrocortisone sodium succinate or hydrocortisone acetate or dexamethasome (optionally DEXTENZA™, OZURDEX™, NEOFORDEX™);
the pharmaceutical composition or a therapeutic combination of drugs as provided herein, or the product of manufacture provided herein, or the kit provided herein, is formulated as, is administered as, or comprises or is manufactured as:
a pharmaceutical formulation, or a dressing or a dermal patch, or an implant,
and optionally the pharmaceutical composition or a therapeutic combination is formulated as: gel, a liquid, an aerosol, a spray, a mist, a lotion, a cream, a powder, a tablet, a capsule, a pill, a geltab, an enema, a suppository, a transdermal or intraoral patch, or an injectable formulation, a subcutaneous, an intramuscular (IM) or intravenous (IV) formulation, to make a pharmaceutical formulation;

the antibiotic, or macrolide drug, optionally doxycycline or azithromycin, is administered by starting with a loading dose, optionally an oral, IV or IM dosage of between about 400 mg to 500 mg and 1 g, or at about 500 mg, optionally with follow up administrations daily every 4 to 10 days, or every 1, 2, 3, 4, 5, 6, 7, 8, 9, 10 days or up to 20 or more days, at a lower dosage of between about 100 gm to 300 mg, or about 250 mg, total daily dosage, optionally continuing for between about one week to one month;
(b) the azithromycin is administered (optionally, ZITHROMAX™, or AZITHROCIN™) at a 500 mg bid loading dose on day one, then 500 mg in the morning (MANE) for days two, three and four, then azithromycin ceased and replaced by doxycycline (optionally, DORYX™, DOXYHEXA™, DOXYLIN™) 100 mg bid for the remainder of the treatment (ten or eleven days); or
(c) the azithromycin is first administered (optionally, ZITHROMAX™, or AZITHROCIN™) at a 500 mg bid loading dose on day one, then 500 mg in the morning (MANE) for days two, three and four, then azithromycin ceased, and doxycycline 100 mg bid (or between about 25 to 500 mg bid) (optionally, DORYX™, DOXYHEXA™, DOXYLIN™) every day for the full duration of the treatment (ten or eleven days, or more);
the administration of the pharmaceutical composition or a therapeutic combination of drugs as provided herein, or the product of manufacture as provided herein, or the kit as provided herein, or treatment, lasts between about 10 days and 3 weeks, or 11 days and 2 weeks, or for about 10, 11, 12, 13 or 14 days, or between about one month and one year; and/or
zinc, optionally zinc sulfate or zinc chelate, is administered at a dosage of 100 mg MANE, or between about 50 mg to 150 mg, every day of the treatment or administration.

In alternative embodiments, provided are transdermal devices comprising or manufactured to have: a plurality of or multiple drug containing or drug storage compartments (or cells or wells), each compartment sealed or having contiguous walls such that when a drug formulation (optionally a liquid, liposome, gel or hydrogel drug formulation) is placed inside one of the plurality of or multiple drug containing or drug storage compartments or cells or wells the liquid or gel drug formulation is contained in the compartment, and the compartment has one side open to a removable (and optionally a flexible) release liner which can be removed by a user, and on the same side of the transdermal device as the open side of (or side covered by the release liner) the compartment or cell or well to the flexible and removable release liner is affixed an adhesive (optionally comprising an acrylate polymer or a pressure-sensitive adhesive (PSA)) to affix the transdermal device to a skin surface, wherein the adhesive is covered until removal of the removable release liner. In alternative embodiments, the transdermal device comprises between 2 and 10 separate, or individual, drug containing or drug storage cells or compartments, and optionally each of the multiple compartments (or cells, or wells) has a storage capacity of between about 5 μl and 1000 ul (or one ml), or between about 20 μl and 500 μl, or between about 50 μl and 5 ml. In alternative embodiments, the transdermal device comprises 3 separate, or individual, drug containing or drug storage cells or compartments.

In alternative embodiments, a first compartment comprises an avermectin class drug (optionally ivermectin), a second compartment comprises an antibiotic (optionally doxycycline or azithromycin) and a third compartment comprises zinc (optionally a zinc chelate such as zinc sulphate, acetate, gluconate or picolinate, or zinc oxide nanoparticles). In alternative embodiments, a first compartment comprises ivermectin, a second compartment comprises doxycycline and a third compartment comprises zinc chelate. In alternative embodiments of the transdermal device as described herein, the first compartment comprises 48 mg of ivermectin, the second compartment comprises 300 mg of doxycycline and the third compartment comprises 50 mg of zinc chelate.

In alternative embodiments, provided are methods for treating, preventing or ameliorating (optionally decreasing the symptoms of, or decreasing the mortality of) a coronavirus infection caused by COVID-19, or a variant, alternative clade or substrain thereof, the method comprising administering to an individual in need thereof the transdermal device as described herein.

In alternative embodiments, provided are transdermal devices as described herein for use in treating, preventing or ameliorating (optionally decreasing the symptoms of, or decreasing the mortality of) a coronavirus infection caused by COVID-19, or a variant, alternative clade or substrain thereof.

In alternative embodiments provided are uses of the transdermal devices as described herein for the manufacture of a medicament for treating, preventing or ameliorating (optionally decreasing the symptoms of, or decreasing the mortality of) a coronavirus infection caused by COVID-19, or a variant, alternative clade or substrain thereof.

The details of one or more exemplary embodiments of the invention are set forth in the description below. Other features, objects, and advantages of the invention will be apparent from the description and drawings, and from the claims.

All publications, patents, patent applications cited herein are hereby expressly incorporated by reference in their entireties for all purposes.

### Description of Embodiments

In alternative embodiments, provided are pharmaceutical compositions and therapeutic combinations of drugs, including products of manufacture and kits, for treating, preventing or ameliorating (for example, decreasing the symptoms of, or decreasing the mortality of) a viral infection, for example, a coronavirus infection such as a COVID-19 or variant thereof, and methods for making and using same. In alternative embodiments, the coronavirus infection is a so-called “long COVID” syndrome, or Post-COVID Conditions (PCC), or post-acute sequelae of SARS COV-2 infection (PASC). In alternative embodiments, provided are products of manufacture, kits and transdermal devices for delivering pharmaceutical compositions and therapeutic combinations of drugs as provided herein.

Although there are active single and double agents that inhibit the growth of Coronavirus, when used alone they have not eradicated the viral infection quickly enough, thus resulting in many Coronavirus-infected patients become ill with a cytokine storm and/or admitted to intensive care, and a small percentage have died.

To address this failing, the inventor has discovered that a combination of at least three drugs or more in correct doses are needed to inhibit adequately the multiplication of the Coronavirus (for example, COVID-19) virus intracellularly to prevent hospitalization and/or death. The inventor discovered drug combinations effective for treating, ameliorating and/or preventing “long COVID” or Post-COVID Conditions (PCC), or post-acute sequelae of SARS CoV-2 infection (PASC). The inventor discovered drug combinations that include metformin (or GLUCOPHAGE™) and/or a 3C-like (3CLpro) protease (also known as main protease (Mpro), also known as C30 endopeptidase or 3-chymotrypsin-like protease) inhibitor such as ensitrelvir (or XOCOVA™), then even a delayed or late start of the treatment can result in a cure. The inventor further discovered that delivering transdermally any drug or drug combination, for example, a drug combinations or formulations as provided herein (for example, metformin and/or a 3C-like (3CLpro) protease inhibitor such as ensitrelvir, with at least one, two, three or more other drugs); or, zinc, ivermectin, and doxycycline (for example ZIVERDOX™) is effective for treating, ameliorating and/or preventing Coronavirus (for example, COVID-19).

### Products of Manufacture and Kits

Provided are products of manufacture and kits for practicing methods as provided herein; and optionally, products of manufacture and kits can further comprise instructions for practicing methods as provided herein.

Provided are compositions, including preparations, formulations and/or kits, comprising combinations of ingredients, for example, pharmaceutical compositions and therapeutic combination of drugs as described herein. In alternative embodiments, pharmaceutical compositions and therapeutic combination of drugs as described herein can be mixed and administered together, or alternatively, they can be an individual member of a packaged combination of ingredients, for example, a liquid component and a solid product component manufactured in a separate compartment, package, kit or container; for example, where all or a subset of the combinations of ingredients are manufactured in a separate compartment, package or container. In alternative aspects, the package, kit or container comprises a blister package, a clamshell, a tray, a shrink wrap and the like.

In one aspect, the package, kit or container comprises a “blister package” (also called a blister pack, or bubble pack). In one aspect, the blister package is made up of two separate elements: a transparent plastic cavity shaped to the product and its blister board backing. These two elements are then joined together with a heat sealing process which allows the product to be hung or displayed. Exemplary types of “blister packages” include: Face seal blister packages, gang run blister packages, mock blister packages, interactive blister packages, slide blister packages.

Blister packs, clamshells or trays are forms of packaging used for goods; thus, provided are for blister packs, clamshells or trays comprising a drug combination or formulation as provided herein, or a drug combination, pharmaceutical preparations or pharmaceutical compositions used to practice methods as provided herein. Blister packs, clamshells or trays can be designed to be non-reclosable, so consumers can tell if a package has already opened. They are used to package for sale goods where product tampering is a consideration, such as the pharmaceuticals as provided herein. In one aspect, a blister pack comprises a moulded PVC base, with raised areas (the “blisters”) to contain the tablets, pills, etc. comprising the combinations of drugs drug combination, or formulations, pharmaceutical preparations or pharmaceutical compositions used in methods as provided herein, covered by a foil laminate.

Tablets, pills, etc. can be removed from the pack either by peeling the foil back or by pushing the blister to force the tablet to break the foil. In one aspect, a specialized form of a blister pack is a strip pack. In one aspect, in the United Kingdom, blister packs adhere to British Standard 8404.

In one embodiment, provided is a method of packaging wherein the compositions comprising combinations of ingredients (for example, pharmaceutical compositions and therapeutic combination of drugs as described herein) are contained in-between a card and a clear PVC. The PVC can be transparent so the item (pill, tablet, geltab, etc.) can be seen and examined easily; and in one aspect, can be vacuum-formed around a mould so it can contain the item snugly and have room to be opened upon purchase. In one aspect, the card is brightly colored and designed depending on the item (pill, tablet, geltab, etc.) inside, and the PVC is affixed to the card using pre-formed tabs where the adhesive is placed. The adhesive can be strong enough so that the pack may hang on a peg, but weak enough so that this way one can tear open the join and access the item. Sometimes with large items or multiple enclosed pills, tablets, geltabs, etc., the card has a perforated window for access. In one aspect, more secure blister packs, for example, for items such as pills, tablets, geltabs, etc. are used, and they can comprise of two vacuum-formed PVC sheets meshed together at the edges, with the informative card inside. These can be hard to open by hand, so a pair of scissors or a sharp knife may be required to open.

In one aspect, blister packaging comprises at least two or three or more components: a thermoformed “blister” which houses multi-ingredient combination as provided herein, and then a “blister card” that is a printed card with an adhesive coating on the front surface. During the assembly process, the blister component, which is most commonly made out of PVC, is attached to the blister card using a blister machine. This machine introduces heat to the flange area of the blister which activates the glue on the card in that specific area and ultimately secures the PVG blister to the printed blister card. The thermoformed PVG blister and the printed blister card can be as small or as large as you would like, but there are limitations and cost considerations in going to an oversized blister card. Conventional blister packs can also be sealed (for example, using an AERGO 8 DUO™, SCA Consumer Packaging, Inc., DeKalb IL) using regular heat seal tooling. This alternative aspect, using heat seal tooling, can seal common types of thermoformed packaging.

In alternative embodiments, pharmaceutical compositions and therapeutic combination of drugs as described herein are formulated, for example, as a powder, for example, as lyophilised material, for example, a lyophilized encapsulated product, for example, for practicing methods as provided herein, can be packaged alone or in combinations, for example, as “blister packages” or as a plurality of packettes, including as lidded blister packages, lidded blister or blister card or packets or packettes, or a shrink wrap.

In alternative embodiments, laminated aluminium foil blister packs are used, for example, for the preparation of therapeutic combinations or formulations as provided herein, or for pharmaceutical preparations or pharmaceutical compositions used in methods as provided herein. Products or kits comprise an aqueous solution(s) which are dispensed (for example, by measured dose) into containers. Trays can be freeze-dried to form tablets which take the shape of the blister pockets. The alufoil laminate of both the tray and lid fully protects any highly hygroscopic and/or sensitive individual doses. In one aspect, the pack incorporates a child-proof peel open security laminate. In one aspect, the system give tablets an identification mark by embossing a design into the alufoil pocket that is taken up by the tablets when they change from aqueous to solid state. In one aspect, individual ‘push-through’ blister packs/packettes are used, for example, using hard temper aluminium or aluminum (for example, alufoil) lidding material. In one aspect, hermetically-sealed high barrier aluminium or aluminum (for example, alufoil) laminates are used. In one aspect, products of manufacture include kits or blister packs, use foil laminations and strip packs, stick packs, sachets and pouches, peelable and non-peelable laminations combining foil, paper, or film for high barrier packaging.

In alternative embodiments, multi-component products of manufacture, including kits or blister packs as provided herein, include memory aids to help remind patients when and how to take the therapeutic combination. This safeguards the therapeutic combination's efficacy by protecting each tablet, geltab or pill until it's taken; gives the product or kit portability, makes it easy to take a dose anytime or anywhere.

### Dosaging and Packaging for Therapeutic or Prophylactic Purposes

In alternative embodiments, provided are pharmaceutical compositions and therapeutic combination of drugs as described herein, and drug combinations and drug delivery devices comprising these combinations for therapeutic and/or prophylactic (prevention) purposes.

In alternative embodiments, a therapeutic or a prophylactic drug or ingredient combination “package”, which can be a blister pack, clamshell, or a nebulizer, inhaler, respirator or CPAP insert, or the like, is designed such that a particular drug or ingredient combination (for example, a drug or ingredient combination have 2, 3, 4, 5, or 6 ingredients or active agents, wherein one, several or all are separately formulated or formulated into one delivery agent such as a capsule or geltab, or nebulizer, inhaler, respirator or CPAP insert), to be taken by a user every day, every other day, every week, every two weeks or every 4 weeks (i.e., monthly). In alternative embodiments, the therapeutic or a prophylactic drug combination “package” is designed (for example, instructing the user) to take the drug combination as a staggered dosage, for example, one administration of the drug combination for two or three days in a row staggered by a week before the next two or three day administration cycle begins again.

### Formulations and Pharmaceutical Compositions

In alternative embodiments, provided are pharmaceutical formulations or compositions for use in in vivo, in vitro or ex vivo methods to treat, prevent, reverse and/or ameliorate a viral infection, for example, coronavirus (optionally COVID-19, optionally drug combinations as provided herein are effective for treating, ameliorating and/or preventing “long COVID” or Post-COVID Conditions (PCC), or post-acute sequelae of SARS COV-2 infection (PASC).

In alternative embodiments, the pharmaceutical compositions as provided herein or used to practice methods as provided herein can be administered parenterally, topically, orally or by local administration, such as by aerosol, mist, powder, intraorally or transdermally. These pharmaceutical compositions can be formulated in any way and can be administered in a variety of unit dosage forms depending upon the condition or disease and the degree of illness, the general medical condition of each patient, the resulting preferred method of administration and the like. Details on techniques for formulation and administration are well described in the scientific and patent literature, see, for example, the latest edition of Remington's Pharmaceutical Sciences, Maack Publishing Co., Easton PA (“Remington's”). For example, in alternative embodiments, pharmaceutical compositions or therapeutic combination of drugs as provided herein are formulated in a buffer, in a saline solution, in a powder, an emulsion, in a vesicle, in a liposome, in a nanoparticle, in a nanolipoparticle and the like. In alternative embodiments, pharmaceutical compositions or therapeutic combination of drugs as provided herein can be formulated in any way and can be applied in a variety of concentrations and forms depending on the desired in vivo, in vitro or ex vivo conditions, a desired in vivo, in vitro or ex vivo method of administration and the like. Details on techniques for in vivo, in vitro or ex vivo formulations and administrations are well described in the scientific and patent literature. Formulations and/or carriers used to practice methods as provided herein can be in forms such as tablets, pills, powders, capsules, liquids, gels, syrups, slurries, suspensions, etc., suitable for in vivo, in vitro or ex vivo applications.

In alternative embodiment, pharmaceutical compositions or therapeutic combination of drugs as provided herein or used to practice methods as provided herein can comprise a solution of compositions disposed in or dissolved in a pharmaceutically acceptable carrier, for example, acceptable vehicles and solvents that can be employed include water and Ringer's solution, an isotonic sodium chloride. In addition, sterile fixed oils can be employed as a solvent or suspending medium. For this purpose any fixed oil can be employed including synthetic mono-or diglycerides, or fatty acids such as oleic acid. In one embodiment, solutions and formulations used to practice the invention are sterile and can be manufactured to be generally free of undesirable matter. In one embodiment, these solutions and formulations are sterilized by conventional, well known sterilization techniques.

The pharmaceutical compositions or therapeutic combination of drugs as provided herein, and solutions and formulations as provided herein, or used to practice methods as provided herein can comprise auxiliary substances as required to approximate physiological conditions such as pH adjusting and buffering agents, toxicity adjusting agents, for example, sodium acetate, sodium chloride, potassium chloride, calcium chloride, sodium lactate and the like. The concentration of active agent in these formulations can vary widely, and can be selected primarily based on fluid volumes, viscosities and the like, in accordance with the particular mode of in vivo, in vitro or ex vivo administration selected and the desired results.

The pharmaceutical compositions or therapeutic combination of drugs as provided herein or used to practice methods as provided herein can be delivered by the use of liposomes. By using liposomes, particularly where the liposome surface carries ligands specific for target cells (for example, an injured or diseased neuronal cell or CNS tissue), or are otherwise preferentially directed to a specific tissue or organ type, one can focus the delivery of the active agent into a target cells in an in vivo, in vitro or ex vivo application.

### Nanoparticles, Nanolipoparticles and Liposomes

Also provided are nanoparticles, nanolipoparticles, vesicles and liposomal membranes comprising pharmaceutical compositions or therapeutic combination of drugs as provided herein or used to practice methods as provided herein. These nanoparticles, nanolipoparticles, vesicles and liposomal membranes can be delivered transdermally to an individual in need thereof using a transdermal delivery device as provided herein.

In alternative embodiments, provided are multilayered liposomes comprising pharmaceutical compositions or therapeutic combination of drugs as provided herein, or as used to practice methods as provided herein, for example, as described in Park, et al., U.S. Pat. Pub. No. 20070082042. The multilayered liposomes can be prepared using a mixture of oil-phase components comprising squalane, sterols, ceramides, neutral lipids or oils, fatty acids and lecithins, to about 200 to 5000 nm in particle size, to entrap a composition used to practice methods as provided herein.

Liposomes can be made using any method, for example, as described in Park, et al., U.S. Pat. Pub. No. 20070042031, including method of producing a liposome by encapsulating an active agent, the method comprising providing an aqueous solution in a first reservoir; providing an organic lipid solution in a second reservoir, and then mixing the aqueous solution with the organic lipid solution in a first mixing region to produce a liposome solution, where the organic lipid solution mixes with the aqueous solution to substantially instantaneously produce a liposome encapsulating the active agent; and immediately then mixing the liposome solution with a buffer solution to produce a diluted liposome solution.

In one embodiment, liposome compositions used to practice methods as provided herein comprise a substituted ammonium and/or polyanions, for example, for targeting delivery of a compound, as described for example, in U.S. Pat. Pub. No. 20070110798.

Provided are nanoparticles comprising pharmaceutical compositions or therapeutic combination of drugs as provided herein, or as used to practice methods as provided herein, in the form of active agent-containing nanoparticles (for example, a secondary nanoparticle), as described, for example, in U.S. Pat. Pub. No. 20070077286. In one embodiment, provided are nanoparticles comprising a fat-soluble active drug or a fat-solubilized water-soluble active agent to act with a bivalent or trivalent metal salt.

In one embodiment, solid lipid suspensions can be used to formulate and to deliver compositions used to practice methods as provided herein to mammalian cells in vivo, in vitro or ex vivo, as described, for example, in U.S. Pat. Pub. No. 20050136121.

### Inhalers, Nebulizers, Puffers and Nasal Sprays

In alternative embodiments, drug delivery devices comprising an inhalation device or inhaler or aerosol or a nasal spray device, for example, a nebulizer, a puffer (as for asthma) or a modified hair dryer and the like, are used for the delivery of a therapeutic combination of drugs, a pharmaceutical dosage form or a formulation as provided herein. In alternative embodiments, provided are methods for administering a therapeutic combination of drugs, a pharmaceutical dosage form or a formulation as provided herein using an inhalation device, nebulizer, puffer or inhaler or a nasal spray device, for example, for the delivery of metformin and/or a 3C-like (3CLpro) protease inhibitor such as ensitrelvir with at least one, two, three or more other drugs.

In alternative embodiments, the inhaler, nebulizer, puffer or the nasal spray device is a hand-held or otherwise portable (for example, worn around the neck) inhaler or a nasal spray device, and optionally the inhaler or a nasal spray device is a metered or dose-counting inhaler or a nasal spray device. In alternative embodiments, the inhaler or the nasal spray device is a device as described in for example, U.S. Pat. Nos. 10,583,261, or 10,561,809 (describing a breath actuated dry powder inhaler with a single air circulation chamber for de-agglomeration of entrained powdered medicament), or U.S. Pat. No. 10,561,807 (describing inhaler devices configured for consuming a defined capacity and generate an aerosol spray, mist, or aerosol imparted with flavor, a sensor configured to detect a predefined variable, an interface configured to make a notification to an inhaler of the aerosol, spray or mist, and a controller), or U.S. Pat. No. 10,463,815 (describing a dry powder inhaler may include a powder storage region, an inlet channel, a dispersion chamber, and an outlet channel); or U.S. patent application publication no. 20200069897 (describing inhalers having a breath actuated trigger mechanism reactive to an inhalation flow to trigger the release of a substance to be inhaled); or 20200061314 (describing a smart inhaler device having a flow pathway comprising a cartridge receptacle that is able to house a cartridge, flow meter, pump, and vaporizer; a wireless communication module; and at least one sensor that captures identifying information related to the cartridge); or 2020004691 (describing dry powder inhalers having replaceable cartridges containing a dry powder for local or systemic delivery through the pulmonary tract and lungs); or 20200046916 (describing an inhaler having a refill assembly comprising: a patient port; a canister actuable by the reusable assembly to deliver a dose of medicament to the patient port, a sleeve which is selectively actuable by a user independently of the reusable assembly so as to act on the canister to deliver a dose of medicament); or 20200046029 (describing an apparatus for generating an aerosol, spray or mist, and/or a vapour in an inhaler device includes a reservoir for storing a supply of a liquid; a heating system fluidly connected with the reservoir for receiving the liquid and configured to heat the liquid to generate the aerosol, spray or mist and/or vapor therefrom; a pumping system configured to pump the liquid from the reservoir to the heating system; and a valve arrangement for regulating flow from the pumping system to the heating system); or 20200016345 (describing a dry powder inhaler having a first chamber having an orifice for holding a dry powder and a gas, and a second chamber directly connected to the first chamber by at least one passageway for receiving an aerosolized form of the dry powder from in the first chamber and delivering the aerosolized dry powder to a user). An inhaler as provided herein, or as used in methods as provided herein, can comprise use of a dose counter, for example, as described in U.S. Pat. No. 10,561,808.

In alternative embodiments, the inhaler or the nasal spray device is a hand-held or otherwise portable inhaler or a nasal spray device is used or intended for use on public transport such as buses, trams, trains, aircraft and/or boats, or in places of commerce such as stores, bars, sporting events, movies theaters, theater, musical events, or any gathering of people.

In alternative embodiments, a drug or drug combination or a formulation as provided herein are delivered as a liquid, powder, spray or a mist through an oxygen tubing or an inhalation device such a CPAP (continuous positive air pressure) device, e.g., as used in sleep apnea treatment, a respirator or an ventilator.

In alternative embodiments, CPAP devices for delivering a drug or drug combination as provided herein can comprise components or be fabricated as, or be used, as described in e.g., U.S. Pat. Nos. 10,595,814; 10,549,057 (describing a ventilator system includes a mask to be placed over a wearer's face); U.S. Pat. No. 10,543,333 (describing a vent arrangement for a mask or associated conduit to discharge exhaled gas from the mask); and/or U.S. Pat. No. 10,406,312 (describing a CPAP flow driver for using nebulizer with CPAP apparatus).

In alternative embodiments, a medical device for inhalation delivery of a drug or a medication or combination as provided herein comprises a dry powder inhaler (such as a dry powder disk inhaler, e.g., as a DISKUS™ device), optionally having a dose counter window so user can see how many doses are left), e.g., where the powder is dose dispensed by (using) a disposable, refillable or replaceable cassette, packette or disk; and the dry powder dispensing can be breath activated, e.g., as an AEROLIZER™, FLEXHALER™, PRESSAIR™, DISKUS™, HANDIHALER™, TWISTHALER™, ELLIPTA™, NEOHALER™, RESPICLICK™, ROTAHALER™ or TUBUHALER™ device.

In alternative embodiments, the anti-viral drug combinations or medications as provided herein (for example, metformin and/or a 3C-like (3CLpro) protease inhibitor such as ensitrelvir, with at least one, two, three or more other drugs) are formulated as a powder (for example, a dry powder), a microparticle or a nanoparticle, or an aerosol, spray or mist. In alternative embodiments, the powder can be an agglomeration of powder particles or an agglomerate having irregular geometries such as width, diameter, and length. In alternative embodiments, the dry powder can be formulated as a granule of a physiologically acceptable excipient to be used as a carrier for a dry powder formulation for inhalation as described for example, in U.S. Pat. No. 10,583,085.

In alternative embodiments, the chloroquine (optionally, ARALEN™), chloroquine phosphate, chloroquine diphosphate, hydroxychloroquine (optionally, PLAQUENIL™) is formulated at between about 50% to 100% concentration as a liquid or aqueous formulation. In alternative embodiments, the chloroquine (optionally, ARALEN™), chloroquine phosphate, chloroquine diphosphate, hydroxychloroquine (optionally, PLAQUENIL™) is formulated and delivered at a dosage regimen of about 0.2 mg/kg to about 150 mg/kg per dose.

In alternative embodiments, methods of delivery of drug combinations as provided herein (for example, metformin and/or a 3C-like (3CLpro) protease inhibitor such as ensitrelvir, with at least one, two, three or more other drugs) comprise treatment regimens where the drug, medication or combination of drugs are administered every hour, every other hour, once, twice, three, four, five, six, seven, eight, nine, ten, eleven or twelve times a day. In alternative embodiments, the length of time of treatment, or the exact dosaging or dosage regimen, is determined by the clinician, or the administration is to begin immediately after possible exposure to an individual having (or exposed to another individual having) a viral infection, for example, a coronavirus infection such as a COVID-19 or variant thereof, or an infection caused by a virus in the subfamily Orthocoronavirinae, or a virus in the family Coronaviridae, or a virus in the order Nidovirales.

In alternative embodiments, the drugs (for example, metformin and/or a 3C-like (3CLpro) protease inhibitor such as ensitrelvir, with at least one, two, three or more other drugs) are formulated at between about 50% to 100% concentration as a liquid or aqueous formulation, which can be used either as an aerosol, spray or mist and/or given orally. In alternative embodiments, the drugs are formulated and delivered (for example, by inhalation and/or orally) at a dosage regimen of about 0.2 mg/kg to about 150 mg/kg per dose.

### Transdermal Drug Delivery Devices

In alternative embodiments, provided are transdermal delivery devices such as bandages, skin patches or dressings for delivering transdermally any drug or drug combination, for example, a drug combinations or formulations as provided herein (for example, metformin and/or a 3C-like (3CLpro) protease inhibitor such as ensitrelvir, with at least one, two, three or more other drugs); or, zinc, ivermectin, and doxycycline (for example ZIVERDOX™).

In alternative embodiments, any drug or drug combination, for example, a drug combination as provided herein, are contained in the transdermal delivery devices such that they can pass through a drug- or formulation-permeable surface of the bandage, skin patch or dressing, where the permeable surface is laid against the skin of an individual in need thereof. In other words, the skin patch or dressing has one surface that is permeable to drug combinations or formulations as provided herein. The drug combinations or formulations can be formulated in the form of a gel or hydrogel.

In alternative embodiments, the surface of the transdermal delivery device as provided herein, for example, the bandage, skin patch or dressing, has an (one) outer surface, layer or covering or laminate (for example, a “release liner”) that is removable by a user (for example, an individual in need thereof) before being place against the skin.

In alternative embodiments, the transdermal delivery device as provided herein, for example, the bandage, skin patch or dressing, adheres to the skin, wherein a skin-adherent composition is positioned around the entire periphery of a drug combination or formulation compartment such that no drug combination or formulation (for example, formulated as a gel or hydrogel) leaks or escapes out of the dressing, but rather is contained between the skin patch or dressing and the skin, thus ensuring that the drug combination or formulation is in contact only with skin under the skin-adherent dressing or patch.

In alternative embodiments, the transdermal delivery device is comprised of acrylate polymers, optionally acrylate polymers that are pressure-sensitive adhesives (PSA), for example, the transdermal delivery device can comprise or be manufactured with, or have adhesives comprising:
an ethylene vinyl acetate co-polymer, as described in U.S. Pat. No. 7,396,976;
Methacrylate copolymers: these copolymers have excellent adhesion and cohesive strength, as well as their ability to form clear and transparent films;
Acrylate-vinyl acetate copolymers: these copolymers have a good balance of adhesion, cohesion, flexibility and durability properties;
Acrylate-acrylonitrile copolymers: these copolymers have high adhesion to a wide range of substrates and good resistance to UV light and weathering;
Acrylate-butadiene copolymers: these copolymers have strong adhesion, good flexibility, and resistance to heat and aging.

In alternative embodiments, the adhesives are contained in microcapsules as described for example in U.S. Pat. No. 7,396,976.

In alternative embodiments, the transdermal delivery devices, for example, bandages, patches or dressings, comprise a skin adhesive, which can be made from a combination of materials to provide the necessary adhesion to the skin, as well as protection and comfort, for example, a transdermal delivery device as provide herein comprises or is manufactured to have:
an adhesive material can be a medical-grade, skin-friendly substance that is able to stick to the skin and hold the bandage in place; for example, a pressure-sensitive adhesive (PSA) made from for example acrylate polymers, as noted above;
backing material to provides a base for the adhesive and protects the wound from further injury, and can comprise materials such as cloth, plastic film, or non-woven fabric;
optionally can also have an absorbent pad made from materials comprising cellulose or hydrocolloid to absorb exudate from the wound;
optionally can also have a release liner, for example, that is made of a paper or plastic film that protects the adhesive until the bandage is ready to be used.

In alternative embodiments, the transdermal delivery devices comprise or use non-irritating adhesives to attach to skin, for example, to human skin, including for example:
Hypoallergenic tape, designed for sensitive skin and is less likely to cause skin irritation or an allergic reaction;
Fabric bandages, which can have a soft, cloth-like backing that is gentle on the skin and less likely to cause irritation;
Hydrocolloid bandages, which can have a gel-like substance that helps to protect and heal the skin, while also providing a non-irritating adhesive,
Silicone adhesive bandages, which can have a silicone-based adhesive that is gentle on the skin and less likely to cause irritation or leave residue.

In alternative embodiments, the transdermal delivery devices as provided herein are manufactured as hydrocolloid dressings or bandages which can made of a combination of materials and chemicals, including for example:
Hydrocolloid material comrpising a gel-forming substance that helps to protect and heal the wound, and can be made from a mixture of polymers, such as pectin or carboxymethylcellulose, and gel-forming agents, such as sodium carboxymethylcellulose or sodium polyacrylate;
Adhesive, which can be a medical-grade, skin-friendly adhesive that is non-irritating and gentle on the skin;
Backing material, which can be a semi-permeable material, such as polyurethane or polyethylene, that allows air and moisture to circulate and helps to prevent the bandage from sticking to the wound.

In alternative embodiments, the transdermal delivery devices as provided herein are manufactured as silicone adhesive dressings or bandages and can be made of the following materials:
Silicone adhesive: can be soft and flexible, which helps to reduce friction and minimize irritation on the skin;
Backing material, can be a soft, flexible material, such as polyurethane, that helps to conform to the skin and minimize irritation;
optionally a wound pad comprising an absorbent material such as cotton or non-woven fabric to help absorb exudates from the wound.

Silicone adhesives used to manufacture transdermal delivery devices as provided herein can comprise a combination of polymer compounds and fillers, for example:
Polydimethylsiloxane (PDMS), a a linear, flexible polymer that is commonly used in silicone adhesives, it provides good adhesion and elasticity, and is also known for its resistance to heat and chemical degradation;
Silicone resins: these are cross-linked silicone polymers that provide strength and stability to the silicone adhesive, they are often used in combination with PDMS to improve the performance of the adhesive;
Silicone adhesives may also contain fillers, such as silica, to improve their physical properties, and activators or catalysts to promote the curing process.

In alternative embodiments, the transdermal delivery devices as provided herein are divided into sections or multiple compartments (or cells, or wells), optionally each containing (storing before delivery) a different drug, for example an exemplary transdermal delivery devices as provided herein is divided into three sections or compartments (or cells or wells), each compartment having one of zinc, ivermectin and doxycycline; or is divided into four sections or compartments, each section or compartment having one of vitamin D, zinc, ivermectin and doxycycline. By having separate sections or compartments the drugs can be formulated differently, for example, each having different dosages, carriers and/or formulations.

In alternative embodiments, each of the multiple compartments (or cells, or wells) has a storage capacity of between about 5 μl and 1000 μl (or one ml), or between about 20 μl and 500 μl.

### Dosaging in Transdermal Devices

In alternative embodiments, drug combinations or formulations, for example, drug combinations or formulations as provided herein, are formulated in higher concentrations in formulations for delivery by transdermal delivery devices as provided herein. In alternative embodiments, drug combinations or formulations are formulated in gels or hydrogels such that the drug active agents in contact with the skin can permeate into or through the skin.

For example, a drug such as metformin, ensitrelvir, avermectin, an antibiotic (for example, doxycycline or azithromycin), zinc or a vitamin (for example, vitamin D and/or C) is formulated or dosaged at 2 times, 5 times, 10 times or 20 times greater (or optionally in between about 2 to 50 times greater) dosages for formulations in the one or more compartments of a transdermal delivery device as provided herein.

For example, in one compartment or cell or well of a transdermal delivery device as provided herein contains or stores an avermectin class drug such as ivermectin, which is dosaged or formulated in the compartment at sufficiently high concentrations to result in an effective delivery to the individual in need thereof (the patient) a dosage of between about 50 to 2000 μgm/kg body weight per day, optionally dosaged at 200 μgm/kg per day, or about 14 mg/day, optionally dosaged at 220, 225, 150, 275 or 300 μgm/kg per day, or optionally dosaged at 12, 14, 16, 18, 20, 22, 24 or 26 mg per day, or optionally dosaged at between about 10 to 30 mg per day.

In alternative embodiments, ivermectin, doxycycline and zinc chelate are each formulated in a higher dosage (ivermectin 48 mg, doxycycline 400 mg and zinc chelate 100 mg) than would be formulated if the same drug combination was to be orally administered (which would be ivermectin 12 mg, doxycycline 100 mg and zinc chelate 25 mg). In one embodiment one compartment or cell or well of a transdermal delivery device as provided herein contains or stores an avermectin class drug such as ivermectin, which is dosaged or formulated in the compartment at sufficiently high concentrations to result in an effective delivery to the individual in need thereof (the patient) a dosage of about 48 mg/day, optionally dosaged at 24, 26, 28, 30, 32, 34, 36, 38, 40, 42, 44, 46, 48, 50, 52, 54, 56, 58 or 60 mg per day, or optionally dosaged at between about 30 to 60 mg per day.

In alternative embodiments, drug combinations or formulations are formulated in a delivery carrier that facilitates transdermal delivery (for example, formulated in a dermal penetration enhancer), for example, drug combinations or formulations in transdermal delivery devices as provided herein are formulated in: a lipid or liposome or lipid vesicle, an azone (optionally a laurocapram), a sulfoxide such as dimethylsulfoxide or DMSO, an alcohol (optionally ethanol, decanol), a polyol (optionally propylene glycol), an alkane, a fatty acid (optionally oleic acid), an ester, an amine or an amide (optionally urea, dimethylacetamide, dimethylformamide and/or a pyrrolidone such as 2-pyrrolidone), a terpene, a cyclodextrin, a surfactant such as a non-ionic or a cationic surfactant, or a chemical penetration enhancer (CPE). In alternative embodiments, the drug combinations or formulations in transdermal delivery devices as provided herein are formulated in a gelling agent with water.

### Pressure Cuffs or Bands

In alternative embodiments, drug combinations or formulations are formulated in a delivery carrier, or transdermal device, or provided are delivery carriers or transdermal devices as provided herein that further comprises a pressure cuff or band, wherein the delivery carrier or transdermal device is set, affixed, glued or sewn onto a cuff or band designed or manufactured to be worn around an arm (or an upper arm or lower arm), or leg, and the cuff or band is designed or manufactured to allow a user to tighten the cuff around the arm or leg, optionally by use of a hook and loop fastener (a lineal fabric strip with tiny hooks that can mate with another fabric strip with smaller loops, attaching temporarily, until pulled apart) (or VELCRO™), or the cuff is designed or manufactured to tighten around the arm or leg by inflation (as is a sphygmomanometer, or a blood pressure cuff), wherein the cuff or band is wrapped around an arm, or upper arm, and inflated, and optionally cuff is manufactured or designed as described in U.S. Pat. Nos. 7,390,301; 7,544,168 (which describes an automatic cuff-based blood-pressure measuring device); U.S. Pat. No. 10,398,325 (which describes a cuff comprising a first bladder having a width, and a length transverse to the width; and the cuff also includes a second bladder connected to the first bladder and a port fluidly connected to at least one of the first and second bladders); or as described in U.S. patent publication no. US20130060147A1 (describing use of a compressible material to tighten a cuff or band).

In alternative embodiments, the cuff or band is manufactured using cotton, nylon or polyester.

### Drugs and Drug Combinations

In alternative embodiments, transdermal delivery devices as provided herein comprise any pharmaceutical composition or therapeutic combinations of drugs, for example, a transdermal delivery device as provided herein comprises one, two, three, four or five or more of any of the following:
(a) (i) metformin (or GLUCOPHAGE™) and/or (ii) a 3C-like (3CLpro) protease (also known as main protease (Mpro), also known as C30 endopeptidase or 3-chymotrypsin-like protease) inhibitor, optionally ensitrelvir (or XOCOVA™), or ensitrelvir fumaric acid, wherein optionally the metformin is formulated or dosaged at between about 100 mg to 4000 mg per unit dosage (optionally, per tablet); or the metformin is formulated or dosage at between about 0.1 ml to 25 ml liquid metformin/day; and, wherein optionally the ensitrelvir or ensitrelvir fumaric acid is formulated or dosaged at between about 100 mg to 500 mg per unit dosage, or at about 100 mg, 125 mg or 150 mg per unit dosage (optionally, per tablet); and
(b) (i) an avermectin class drug, or ivermectin (or STROMECTOL™); (ii) an avermectin class drug, or ivermectin and an antibiotic (optionally doxycycline or azithromycin); (iii) an avermectin class drug, or ivermectin; an antibiotic (optionally doxycycline or azithromycin), and zinc, and optionally also vitamin D or cholecalciferol, optionally with or without vitamin C;
(c) an antibiotic, or ivermectin; and, an antibiotic (optionally doxycycline or azithromycin), at least one vitamin and zinc;
(d) opaganib or YELIVA™;
(e) opaganib and: an avermectin class drug, or ivermectin; an antibiotic (optionally doxycycline or azithromycin), and zinc, and optionally also vitamin D or cholecalciferol;
(f) plitidepsin (also known as dehydrodidemnin B), or APLIDIN™;
(g) plitidepsin and: an avermectin class drug, or ivermectin; an antibiotic (optionally doxycycline or azithromycin), and zinc, and optionally also vitamin D or cholecalciferol, and optionally also vitamin D or cholecalciferol;
(h) lopinavir, ritonavir (or NORVIR™) and/or oseltamivir,
(i) lopinavir, ritonavir and/or oseltamivir, and: an avermectin class drug, or ivermectin; an antibiotic (optionally doxycycline or azithromycin), and zinc, and optionally also vitamin D or cholecalciferol;
(j) nirmatrelvir and ritonavir (or PAXLOVID™),
(k) nirmatrelvir and ritonavir (or PAXLOVID™), and: an avermectin class drug, or ivermectin; an antibiotic (optionally doxycycline or azithromycin), and zinc, and optionally also vitamin D or cholecalciferol;
(l) chloroquine (optionally, ARALEN™), chloroquine phosphate, chloroquine diphosphate, hydroxychloroquine (optionally, PLAQUENIL™);
(m) chloroquine, chloroquine phosphate, chloroquine diphosphate, hydroxychloroquine; and: an avermectin class drug, or ivermectin; an antibiotic (optionally doxycycline or azithromycin), and zinc, and optionally also vitamin D or cholecalciferol;
(n) abacavir, acyclovir optionally, (ACICLOVIR™), adefovir, amantadine, ampligen, amprenavir (optionally, AGENERASE™), aprepitant, arbidol, atazanavir, atripla, balavir, baloxavir marboxil (XOFLUZA™), bepotastine, bevirimat, bictegravir, biktarvy, brilacidin, cidofovir, caspofungin, lamivudine and zidovudine (optionally, COMBVIR™), cobicstat, colisitin, cocaine, delavirdine, descovy, didanosine, docosanol, dolutegravir, ecoliever, edoxudine, elvitegravir, enfuvirtide, entecavir, epirubicin, epoprostenol, etravirine, famciclovir, fomivirsen, fosamprenavi, foscarnet, fosfonet, galidesivir, ibacitabine, icatibant, idoxuridine, ifenprodil, imiquimod, imunovir, indinavir, inosine, an interferon (optionally interferon type I, interferon type II and/or interferon type III), lamivudine, loviride, ledipasvir, leronlimab, maraviroc, methisazone, moroxydine, nexavir, norvir, a nucleoside analogue (optionally brincidofovir, didanosine, favipiravir (also known as T-705, avigan, or favilavir, Toyama Chemical, Fujifilm, Japan), vidarabine, galidesivir (optionally, BCX4430, IMMUCILLIN-A™), cytarabine, gemcitabine, lamivudine, zalcitabine, abacavir, acyclovir, entecavir, stavudine, telbivudine, zidovudine, idoxuridine and/or trifluridine or any combination thereof), oseltamivir (or TAMIFLU™), peginterferon alfa-2a, penciclovir, peramivir (optionally, RAPIVAB™), perfenazine, pleconaril, plurifloxacin, podophyllotoxin, pyramidine, raltegravir, rifampicin, rilpivirine, rimantadine, ritonavir, saquinavir, sofosbuvir, stavudine, telaprevir, tegobuv, tipranavir, trifluridine, trizivir, tromantadine, truvada, valaciclovir (optionally, VALTREX™), valganciclovir, valrubicin, vapreotide, vicriviroc, vidarabine, viramidine, velpatasvir, vivecon, zalcitabine, zanamivir (optionally, RELENZA™), zidovudine, or any combination thereof;
(o) any one or more drug or drugs in (xiv); and: an avermectin class drug, or ivermectin; an antibiotic (optionally doxycycline or azithromycin), and zinc, and optionally also vitamin D or cholecalciferol;
(p) molnupiravir, emtricitabine (or EMTRIVA™), efavirenz (or SUSTIVA™) or tenofovir; or molnupiravir with emtricitabine and tenofovir (or ATRIPLA™);
(q) molnupiravir, emtricitabine, efavirenz or tenofovir, and: an avermectin class drug, or ivermectin; an antibiotic (optionally doxycycline or azithromycin), and zinc, and optionally also vitamin D or cholecalciferol;
(r) zanamivir (or RELENZA™);
(s) zanamivir, and: an avermectin class drug, or ivermectin; an antibiotic (optionally doxycycline or azithromycin), and zinc, and optionally also vitamin D or cholecalciferol;
(t) efavirenz (optionally SUSTIVA™);
(u) efavirenz, and: an avermectin class drug, or ivermectin; an antibiotic (optionally doxycycline or azithromycin), and zinc, and optionally also vitamin D or cholecalciferol;
(v) nelfinavir (VIRACEPT™) or oseltamivir (or TAMIFLU™),
(w) nelfinavir (VIRACEPT™) or oseltamivir (or TAMIFLU™), and: an avermectin class drug, or ivermectin; an antibiotic (optionally doxycycline or azithromycin), and zinc, and optionally also vitamin D or cholecalciferol;
(x) a thiazolide class drug, optionally nitazoxanide (optionally ALINIA™, or NIZONIDE™) or tizoxanide (or 2-Hydroxy-N-(5-nitro-2-thiazolyl)benzamide);
(y) a thiazolide class drug, optionally nitazoxanide, and: an avermectin class drug, or ivermectin; an antibiotic (optionally doxycycline or azithromycin), and zinc, and optionally also vitamin D or cholecalciferol;
(z) remdesivir (optionally, GS-5734™, Gilead Sciences, or VEKLURY™);
(aa) molnupiravir (or LAGEVRIO™);
(bb) molnupiravir, and: an avermectin class drug, or ivermectin; an antibiotic (optionally doxycycline or azithromycin), and zinc, and optionally also vitamin D or cholecalciferol;
(cc) nevirapine (or VIRAMUNE™);
(dd) nevirapine (or VIRAMUNE™), and: an avermectin class drug, or ivermectin; an antibiotic (optionally doxycycline or azithromycin), and zinc, and optionally also vitamin D or cholecalciferol;
(ee) tenofovir alafenamide, tenofovir disoproxil or tenofovir;
(ff) tenofovir alafenamide, tenofovir disoproxil or tenofovir, and: an avermectin class drug, or ivermectin; an antibiotic (optionally doxycycline or azithromycin), and zinc, and optionally also vitamin D or cholecalciferol;
(gg) salamectin (or SELEHOLD™, REVOLT™, SELARID™ or SENERGY™);
(hh) salamectin, and: an avermectin class drug, or ivermectin; an antibiotic (optionally doxycycline or azithromycin), and zinc, and optionally also vitamin D or cholecalciferol;
(ii) an inhibitor of an S-phase kinase-associated protein 2 (SKP2), or dioscin, or niclosamide, or NICLOCIDE™, FENASAL™, or PHENASAL™,
(jj) an inhibitor of an S-phase kinase-associated protein 2 (SKP2), or dioscin, or niclosamide, and: an avermectin class drug, or ivermectin; an antibiotic (optionally doxycycline or azithromycin), and zinc, and optionally also vitamin D or cholecalciferol;
(kk) ribavirin or tribavirin (or COPEGUS™, REBETOL™, or VIRAZOLE™);
(ll) ribavirin or tribavirin, and: an avermectin class drug, or ivermectin; an antibiotic (optionally doxycycline or azithromycin), and zinc, and optionally also vitamin D or cholecalciferol;
(mm) a viral, or a coronavirus or a COVID-19, protease inhibitor, optionally ASC09 (CAS registry no. 1000287-05-7) (Janssen Research and Development, LLC), ritonavir or ASC09 and ritonavir, or a JAK1/2 inhibitor (optionally baricitinib), optionally compound 11r (University of Lubeck, Germany, see optionally, Zhang et al J. Med Chem 2020, Feb. 11, 2020), or darunavir (or PREZISTA™), cobicistat (or TYBOST™) or darunavir and cobicistat;
(nn) a viral, or a coronavirus or a COVID-19, protease inhibitor, ritonavir, a JAK1/2 inhibitor (optionally baricitinib), or darunavir (or Prezista™) or cobicistat, and: an avermectin class drug, or ivermectin; an antibiotic (optionally doxycycline or azithromycin), and zinc, and optionally also vitamin D or cholecalciferol;
(oo) an mucolytic therapy or drug, optionally acetylcysteine, ambroxol, bromhexine, carbocisteine, erdosteine, mecysteine or dornase alfa, or an expectorant, optionally guaifenesin;
(pp) an mucolytic therapy or drug, and: an avermectin class drug, or ivermectin; an antibiotic (optionally doxycycline or azithromycin), and zinc, and optionally also vitamin D or cholecalciferol;
(qq) an angiotensin-converting enzyme 2 (ACE2) inhibitor, optionally captopril, enalapril, lisinopril, benazepril, fosinopril, quinapril, ramipril, perindopril, moexipril or trandolapril;
(rr) an angiotensin-converting enzyme 2 (ACE2) inhibitor, and: an avermectin class drug, or ivermectin; an antibiotic (optionally doxycycline or azithromycin), and zinc, and optionally also vitamin D or cholecalciferol;
(ss) an anti-vascular endothelial growth factor (VEGF) (optionally VEGF-A) drug or antibody, optionally bevacizumab (or AVASTIN™);
(tt) an anti-vascular endothelial growth factor (VEGF) drug or antibody or bevacizumab and: an avermectin class drug, or ivermectin; an antibiotic (optionally doxycycline or azithromycin), and zinc, and optionally also vitamin D or cholecalciferol;
(uu) a protease inhibitor, optionally danoprevir, optionally a serine protease inhibitor, optionally camostat (or FOIPAN™) or narlaprevir (optionally ARLANSA™)
(vv) a protease inhibitor, danoprevir, a serine protease inhibitor, camostat or narlaprevir, and: an avermectin class drug, or ivermectin; an antibiotic (optionally doxycycline or azithromycin), and zinc, and optionally also vitamin D or cholecalciferol;
(ww) anti-PD-1 checkpoint inhibitor, optionally camrelizumab, or a PD-1 inhibitor (or cemiplimab, dostarlimab, pembrolizumab or nivolumab) or a PD-L1 inhibitor (or atezolizumab, avelumab, cosibelimab or durvalumab);
(xx) anti-PD-1 checkpoint inhibitor, or camrelizumab, or a PD-1 inhibitor or a PD-L1 inhibitor, and: an avermectin class drug, or ivermectin; an antibiotic (optionally doxycycline or azithromycin), and zinc, and optionally also vitamin D or cholecalciferol;
(yy) thalidomide, or thalidomide and glucocorticoid (optionally low-dose glucocorticoid), or and thalidomide and celecoxib;
(zz) thalidomide, or thalidomide and glucocorticoid (optionally low-dose glucocorticoid), and: an avermectin class drug, or ivermectin; an antibiotic (optionally doxycycline or azithromycin), and zinc, and optionally also vitamin D or cholecalciferol;
(aaa) a compound or antibody capable of binding complement factor C5 and blocking membrane attack complex formation, or eculizumab;
(bbb) a compound or antibody capable of binding complement factor C5 and blocking membrane attack complex formation, or eculizumab, and: an avermectin class drug, or ivermectin; an antibiotic (optionally doxycycline or azithromycin), and zinc, and optionally also vitamin D or cholecalciferol;
(ccc) a cathepsin inhibitor, optionally a cathepsin K, B or L inhibitor, or relacatib or balicatib;
(ddd) a cathepsin inhibitor, or relacatib or balicatib, and: an avermectin class drug, or ivermectin; an antibiotic (optionally doxycycline or azithromycin), and zinc, and optionally also vitamin D or cholecalciferol;
(eee) a synthetic nucleoside analog or derivative, or N4-hydroxycytidine, or a prodrug of N4-hydroxycytidine, optionally molnuvpiravir (Merck), or favipiravir (also known as T-705 or AVIGAN™), or favilavir, Toyama Chemical, Fujifilm, Japan, or FABIFLU™ (Glenmark Pharmaceuticals), optionally dosaged at 800 mg bid;
(fff) a synthetic nucleoside analog or derivative, and: an avermectin class drug, or ivermectin; an antibiotic (optionally doxycycline or azithromycin), and zinc, and optionally also vitamin D or cholecalciferol,
wherein the synthetic nucleoside analog or derivative, or N4-hydroxycytidine, or the prodrug of N4-hydroxycytidine, optionally molnuvpiravir or favipiravir, is given as between about 10 mg to 3 gm per dose, or between about 10 mg to 3 gm per day, or can be dosed either as a single dose or given one, two, three or four times a day, or is administered at 200 to 800 mg twice daily, or 200, 400, 600 or 800 mg twice daily, or at 200 to 800 mg three times a day, or at 200, 400, 600 or 800 mg three times a day, or is administered at 200 to 800 mg three times a day for between about 2 to 15 days, or for about 2, 3, 4, 5, 6, 7, 8, 9, 10, 11 or 12 days, and optionally when combined with other drugs a lower dosage is used, optionally administered at 100 or 200 mg three times a day for between about 5 to 15 days, or for about 7, 8, 9, 10, 11 or 12 days;
(ggg) an anti-androgen drug, and optionally the anti-androgen drug is bicalutamide, or CASODEX™, or dutasteride (or AVODART™), and optionally the anti-androgen drug comprises a 5α-reductase inhibitor, and optionally the 5α-reductase inhibitor comprises finasteride (or PROSCAR™, PROPECIA™, or FINIDE™);
(hhh) an anti-androgen drug, and optionally the anti-androgen drug is bicalutamide, or CASODEX™, or dutasteride (or AVODART™), and: an avermectin class drug, or ivermectin; an antibiotic (optionally doxycycline or azithromycin), and zinc, and optionally also vitamin D or cholecalciferol;
(iii) an anti-malarial drug, wherein optionally the anti-malarial drug comprises mefloquine (or Lariam™, Mephaquin™, or Mefliam™), wherein optionally the mefloquine is formulated for oral administration, optionally in tablet or capsule form, optionally as 200 mg, 250 mg or 300 mg tablets;
(jjj) an anti-malarial drug, and: an avermectin class drug, or ivermectin; an antibiotic (optionally doxycycline or azithromycin), and zinc, and optionally also vitamin D or cholecalciferol;
(lll) a peroxisome proliferator-activated receptor (PPAR) agonist, wherein optionally the PPAR agonist comprises fenofibrate, or TRICOR™, FENOBRAT™, FENOGLIDE™ or LIPOFEN™, optionally the PPAR agonist comprises a combination of fenofibrate and pravastatin, or PRAVAFENIX™, or the PPAR agonist comprises bezafibrate, or BEZALIP™, or combination of bezafibrate and chenodeoxycholic acid, or HEPACONDA™, or aluminium clofibrate, or alfibrate, or ciprofibrate, or clinofibrate or LIPOCLIN™, or clofibrate or ATROMID-S™, or clofibride, or gemfibrozil or LOPID™, or ronifibrate, or simfibrate or Cholesolvin™, or any combination thereof,
(mmm) a peroxisome proliferator-activated receptor (PPAR) agonist, and: an avermectin class drug, or ivermectin; an antibiotic (optionally doxycycline or azithromycin), and zinc, and optionally also vitamin D or cholecalciferol;
(nnn) an acetaldehyde dehydrogenase inhibitor, optionally disulfiram, or ANTABUS™, or ANTABUSE™, optionally formulated as an extended, sustained or slow-release disulfiram formulation, optionally the extended, sustained or slow-release disulfiram is formulated as a tablet, a capsule or in an injectable, amphiphilic, absorbable, depot-forming drug delivery system (DDS),
(ooo) an acetaldehyde dehydrogenase inhibitor, and: an avermectin class drug, or ivermectin; an antibiotic (optionally doxycycline or azithromycin), and zinc, and optionally also vitamin D or cholecalciferol;
(ppp) a nicotinic antagonist, a dopamine agonist or a noncompetitive N-Methyl-D-aspartate (NMDA) antagonist, optionally amantadine, or Gocovri™, or Symadine™, or Symmetrel™, optionally dosaged at between about 100 to 200 mg per dose, optionally formulated as tablets or capsules,
(qqq) a nicotinic antagonist, a dopamine agonist or a noncompetitive N-Methyl-D-aspartate (NMDA) antagonist, and: an avermectin class drug, or ivermectin; an antibiotic (optionally doxycycline or azithromycin), and zinc, and optionally also vitamin D or cholecalciferol;
(rrr) a mitochondrial sensitizer, optionally proguanil or chlorguanide (or PALUDRINE™), or a malarial cytochrome bc1 complex inhibitor, optionally atovaquone (or MEPRON™), or a combination of proguanil and atovaquone (or MALARONE™),
and optionally the proguanil, atovaquone or the combination of proguanil and atovaquone are formulated for oral administration, optionally as tablets, optionally the unit dosage of atovaquone is 250 mg, 300 mg, 350 mg, 400 mg, 500 mg or 1 gram, and the unit dosage of proguanil is 100 mg, 250 mg, 300 mg, 350 mg or 400 mg;
(sss) a mitochondrial sensitizer or a malarial cytochrome bel complex inhibitor, and: an avermectin class drug, or ivermectin; an antibiotic (optionally doxycycline or azithromycin), and zinc, and optionally also vitamin D or cholecalciferol;
(ttt) a dendrimer, optionally astodrimer sodium (Starpharma, Melbourne, Australia);
(uuu) a dendrimer, and: an avermectin class drug, or ivermectin; an antibiotic (optionally doxycycline or azithromycin), and zinc, and optionally also vitamin D or cholecalciferol;
(vvv) an antihistamine class drug, optionally azelastine, or Astelin™, Optivar™, Allergodil™, bepotastine (or TALION™, BEPREVE™), brompheniramine, fexofenadine or ALLEGRA™, pheniramine or AVIL™, or chlorpheniramine;
(www) an antihistamine class drug, and: an avermectin class drug, or ivermectin; an antibiotic (optionally doxycycline or azithromycin), and zinc, and optionally also vitamin D or cholecalciferol;
(xxx) a selective serotonin reuptake inhibitor (SSRI) class drug, optionally fluvoxamine, or Luvox™, Faverin™, Fluvoxin™; a peroxisome proliferator-activated receptor (PPAR) agonist, wherein optionally the PPAR agonist comprises fenofibrate, or TRICOR™, FENOBRAT™, FENOGLIDE™ or LIPOFEN™, optionally the PPAR agonist comprises a combination of fenofibrate and pravastatin, or PRAVAFENIX™, or the PPAR agonist comprises bezafibrate, or BEZALIP™, or combination of bezafibrate and chenodeoxycholic acid, or HEPACONDA™, or aluminium clofibrate, or alfibrate, or ciprofibrate, or clinofibrate or LIPOCLIN™, or clofibrate or ATROMID-S™, or clofibride, or gemfibrozil or LOPID™, or ronifibrate, or simfibrate or Cholesolvin™, or any combination thereof;
(yyy) a selective serotonin reuptake inhibitor (SSRI) class drug, and: an avermectin class drug, or ivermectin; an antibiotic (optionally doxycycline or azithromycin), and zinc, and optionally also vitamin D or cholecalciferol;
(zzz) clofazimine, or LAMPENE™, optionally dosaged at about 100 mg per day, or between about 50 mg and 150 mg per day, and/or colchicine (or Colcrys™, Mitigare™), optionally dosaged at about 0.1 mg to 5 mg per day;
(aaaa) clofazimine or colchicine, and: an avermectin class drug, or ivermectin; an antibiotic (optionally doxycycline or azithromycin), and zinc, and optionally also vitamin D or cholecalciferol;
(bbbb) a selective estrogen receptor modulator (SERM), or toremifene (or Fareston™), or clomifene or clomiphene (or CLOMID™, SEROPHENE™);
(cccc) a selective estrogen receptor modulator (SERM), or toremifene or clomifene or clomiphene, and: an avermectin class drug, or ivermectin; an antibiotic (optionally doxycycline or azithromycin), and zinc, and optionally also vitamin D or cholecalciferol;
(dddd) an alpha-ketoamide (α-ketoamide), wherein optionally the alpha-ketoamide is a structure as described by Zhang et al, J. Med. Chem. 2020, 63, 9, 4562-4578, or Meng et al Chem. Sci. (2019) vol. 10, pg 5156 (optionally the structure KAM-2);
(eeee) an alpha-ketoamide, and: an avermectin class drug, or ivermectin; an antibiotic (optionally doxycycline or azithromycin), and zinc, and optionally also vitamin D or cholecalciferol;
(ffff) an acylsulfonamide inhibitor of the NS3-4A serine protease, or paritaprevir, or ombitasvir, or paritaprevir and ombitasvir with ritonavir (TECHNIVIE™);
(gggg) paritaprevir (or Veruprevir™), or ombitasvir, or paritaprevir and ombitasvir with ritonavir, and: an avermectin class drug, or ivermectin; an antibiotic (optionally doxycycline or azithromycin), and zinc, and optionally also vitamin D or cholecalciferol;
(hhhh) at least one vitamin, and optionally the at least one vitamin comprises: vitamin B3 (or pyridine-3-carboxylic acid, niacin or nicotinic acid, or vitamin B3 or niacin administered as a slow release form (or Niaspan FCT™), vitamin D (optionally D2, or ergocalciferol), or Vitamin D3 or cholecalciferol, optionally administered at about 1000 to 4000 ugm/day; vitamin B12, vitamin B6 (or pyridoxine); vitamin K; vitamin A; vitamin E; and/or, vitamin C (optionally administered at 500 mg bid);
(iiii) at least one vitamin, and: an avermectin class drug, or ivermectin; an antibiotic (optionally doxycycline or azithromycin), and zinc;
(jjjj) a compound, drug or formulation that decreases stomach acid production or decreases stomach pH, wherein optionally the compound, drug or formulation comprises famotidine, or PEPCID™, and optionally the famotidine is administered at a dosage of between about 10 to 60 mg per day, or between about 20 to 40 mg per day;
(kkkk) famotidine, and: an avermectin class drug, or ivermectin; an antibiotic (optionally doxycycline or azithromycin), and zinc;
(llll) an immunosuppressive drug, wherein optionally the immunosuppressive drug comprises tocilizumab or atlizumab, or Actemra™, or RoActemra™, or a calcineurin inhibitor (CNI), wherein the CNI comprises ciclosporin (or cyclosporine or cyclosporin), or Neoral™, or Sandimmune™, or tacrolimus, or Protopic™, or Prograf™;
(mmmm) an immunosuppressive drug or a calcineurin inhibitor, and: an avermectin class drug, or ivermectin; an antibiotic (optionally doxycycline or azithromycin), and zinc;
(nnnn) an anti-inflammatory therapy or at least one anti-inflammatory therapy drug, wherein optionally the anti-inflammatory therapy or drug comprises: a sphingosine kinase-2 (SK2) selective inhibitor (optionally, opaganib (optionally, YELIVA™), sirolimus, a JAK1/2/TYK2 inhibitor (optionally ruxolitinib), an anti-CD47 mAb (optionally meplazumab), a cyclooxygenase (COX) (optionally, COX2) inhibitor, a glucocorticoid (optionally a synthetic glucocorticoid, hydrocortisone, dexamethasone (or Dextenza™, Ozurdex™, or Neofordex™) or cortisol, or CORTEF™), plitidepsin or dehydrodidemnin B, or Aplidin™; and/or
(oooo) an anti-inflammatory therapy or at least one anti-inflammatory therapy drug, and: an avermectin class drug, or ivermectin; an antibiotic (optionally doxycycline or azithromycin), and zinc;
wherein optionally the avermectin class drug comprises: ivermectin (or Stromectol™), moxidectin (or CYDECTIN™, EQUEST™, QUEST™), selamectin (or STRONGHOLD™), a milbemycin (optionally milbemectin, milbemycin oxime, moxidectin or nemadectin), doramectin (or DECTOMAX™), eprinomectin or abamectin,
and optionally the avermectin class drug or ivermectin is dosaged for administration at between about 50 to 2000μgm/kg body weight per day, optionally dosaged at 200 μgm/kg per day, or about 14 mg/day, optionally dosaged at 220, 225, 150, 275 or 300 μgm/kg per day, or optionally dosaged at 12, 14, 16, 18, 20, 22, 24 or 26 mg per day, or optionally dosaged at between about 10 to 30 mg per day,
and optionally the avermectin class drug or ivermectin is dosaged for administration at between about 5 to 480 or between about 3 to 500 mg per day,
and optionally the avermectin class drug (optionally ivermectin) is formulated or dosaged at between about 15 to 150 mg/kg, or is about 18, 24, 30, 35, 40, 35, 50, 55, 60, 65, 70, 75, 80, 85, 90, 95, 100, 110 or 120 or more mg/kg,
and optionally the antibiotic comprises a tetracycline class drug, and optionally the tetracycline class drug comprises tetracycline, doxycycline, chlortetracycline, oxytetracycline, glycylcycline, fluorocycline or demeclocycline, tetracycline or Sumycin™; chlortetracycline or Aureomycin™; oxytetracycline; demeclocycline or Declomycin™, Declostatin™, Ledermycin™, Bioterciclin™, Deganol™, Deteclo™, Detravis™, Meciclin™, Mexocine™, Clortetrin™; lymecycline; meclocycline; metacycline; minocycline or MINOCIN™; rolitetracycline; doxycycline, or Doryx™, Doxyhexa™, Doxylin™; tigecycline or TYGACIL™; eravacycline or XERAVA™; sarecycline or SEYSARA™; omadacycline or Nuzyra™; or any combination thereof,
and optionally the therapeutic combination comprises about 25 mg to about 600 mg of the tetracycline class drug, or tetracycline, doxycycline, chlortetracycline, oxytetracycline or demeclocycline,
and optionally the antibiotic comprises a macrolide drug, and optionally the macrolide drug comprises azithromycin, optionally dosaged at between about 50 mg to about 2000 mg per dose or per day (optionally, Zithromax™, or Azithrocin™, optionally an oral extended- or delayed-release formulation of azithromycin, or ZMAX™), clarithromycin (optionally, biaxin™), erythromycin (optionally, Erythrocin™), or fidaxomicin (optionally, Dificid™ or Dificlir™), troleandomycin (optionally, Tekmisin™), tylosin (optionally, Tylocine™ or Tylan™), solithromycin (optionally, Solithera™), oleandomycin (or Sigmamycine™), midecamycin, roxithromycin, kitasamycin or turimycin, josamycin, carbomycin or magnamycin, and/or spiramycin,
and optionally the antibiotic comprises azithromycin, and optionally the therapeutic combination comprises between about 50 mg to about 2000 mg azithromycin, or optionally the azithromycin comprise an oral extended-release formulation of azithromycin,
and optionally the antibiotic comprises a nitroimidazole, or a 2-, 4- and/or 5-nitroimidazole, or a 5-nitro nitroimidazole comprising metronidazole, tinidazole, nimorazole, dimetridazole, pretomanid, ornidazole, megazol and/or azanidazole, or a 2-nitromidazoles comprising benznidazole,
and optionally the antibiotic comprises a β-lactam antibiotic such as penicillin class drug or a cephalosporin class drug,
and optionally the cephalosporin class drug comprises cefalexin or cephalexin, cefadroxil or cefadroxyl, cefazolin or cephazolin, cefapirin or cephapirin, cefalotin or cephalothn, cefradine or corcephradine, cefprozil or cefproxil, cefuroxime, ceftolozane, cefonicid or cefaclor,
and optionally the penicillin class drug comprises penicillin V, penicillin G, cloxacillin, dicloxacillin, flucloxacillin, methicillin, nafcillin, oxacillin, ampicillin, amoxicillin, pivampicillin, bacampicillin, metampicillin, talampicillin, hetacillin, carbenicillin, ticarcillin, temocillin, mezlocillin, piperacillin, azlocillin, clavulanic acid, sulbactam or tazobactam,
and optionally the antibiotic comprises a quinolone class antibiotic such as a fluoroquinolone, or comprises ciprofloxacin, garenoxacin, gatifloxacin, gemifloxacin, levofloxacin or moxifloxacin,
and optionally the antibiotic comprises an ansamycin class drug, or rifampicin, also known as rifampin, bedaquiline,
and optionally the antibiotic comprises artemisinin, amodiaquine, proguanil, sulfadoxine, sulfamethoxypyridazine, pyrimethamine, linezolid, quinine, quinidine, cinchonine, cinchonidine (or quinimax), hydroxychloroquine or chloroquine,
and optionally the at least one vitamin comprises vitamin D or cholecalciferol, or vitamin C, or vitamin D or cholecalciferol and vitamin C, and vitamin D or cholecalciferol is dosaged at between about 3,000 to about 100,000 units vitamin D or cholecalciferol, and optionally the therapeutic combination comprises between about 10,000 to about 50,000 units vitamin D or cholecalciferol, and optionally the vitamin comprises vitamin C, and optionally the vitamin C is formulated or administered at a dosage of between about 500 to 5000 units (U) per dose,
and optionally the zinc comprises or is formulated as: a zinc salt, a zinc chelate, a zinc sulphate, a zinc acetate, a zinc gluconate or a zinc picolinate, and optionally the therapeutic combination comprises between about 1 mg to 250 mg zinc, zinc sulphate, zinc acetate, zinc gluconate or zinc picolinate, or zinc oxide nanoparticles, optionally at a dosage of between about 1 mg to 250 mg,
and optionally the pharmaceutical composition or a therapeutic combination further comprises copper, optionally administered or formulated at a dosage of between about 1 to 200 mg per day, wherein optionally the copper is administered or formulated as cupric chloride and administered intravenously formulated at about 0.4 mg/ml;
and optionally the pharmaceutical composition or a therapeutic combination further comprises selenium, optionally administered as selenious acid formulated at about 65.4 mcg/ml (or μ/ml), and optionally the selenium is administered at a dosage of between about 50 to 100 μ/ml, optionally between about 60 to 100 μgm per day is administered to an adult, and only up to 60 μgm per day for pediatric patients.

In one embodiment the transdermal delivery device as provided herein comprises a first compartment comprising an avermectin class drug (optionally ivermectin), a second compartment comprising an antibiotic (optionally doxycycline or azithromycin) and a third compartment comprising zinc (optionally a zinc chelate such as zinc sulphate, acetate, gluconate or picolinate, or zinc oxide nanoparticles). In one embodiment the transdermal delivery device as provided herein comprises a first compartment comprising ivermectin, a second compartment comprising doxycycline and a third compartment comprising zinc chelate. In one embodiment the transdermal delivery device as provided herein comprises a first compartment comprising between about 24 mg to about 150 mg of ivermectin, a second compartment comprising between about 25 mg to about 600 mg of doxycycline and a third compartment comprising between about 1 mg to 250 mg zinc. In one embodiment the transdermal delivery device as provided herein comprises a first compartment comprising between about 30 mg to about 60 mg of ivermectin, a second compartment comprising between about 100 mg to about 500 mg of doxycycline and a third compartment comprising between about 25 mg to 125 mg zinc. In one embodiment the transdermal delivery device as provided herein comprises a first compartment comprising between about 40 mg to about 50 mg of ivermectin, a second compartment comprising between about 200 mg to about 450 mg of doxycycline and a third compartment comprising between about 50 mg to 100 mg zinc. In one embodiment the transdermal delivery device as provided herein comprises a first compartment comprising 48 mg of ivermectin, a second compartment comprising 300 mg doxycycline and a third compartment comprising 50 mg zinc chelate.

Any of the above aspects and embodiments can be combined with any other aspect or embodiment as disclosed here in the Summary and/or Detailed Description sections.

As used in this specification and the claims, the singular forms “a,” “an” and “the” include plural referents unless the context clearly dictates otherwise.

Unless specifically stated or obvious from context, as used herein, the term “or” is understood to be inclusive and covers both “or” and “and”.

Unless specifically stated or obvious from context, as used herein, the term “about” is understood as within a range of normal tolerance in the art, for example within 2 standard deviations of the mean. About (use of the term “about”) can be understood as within 20%, 19%, 18%, 17%, 16%, 15%, 14%, 13%, 12% 11%, 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Unless specifically stated or obvious from context, as used herein, the terms “substantially all”, “substantially most of”, “substantially all of” or “majority of” encompass at least about 75%, 80%, 85%, 90%, 91%, 92%, 93%, 94%, 95%, 96%, 97%, 98%, 99% or 99.5%, or more of a referenced amount of a composition.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents. Incorporation by reference of these documents, standing alone, should not be construed as an assertion or admission that any portion of the contents of any document is considered to be essential material for satisfying any national or regional statutory disclosure requirement for patent applications. Notwithstanding, the right is reserved for relying upon any of such documents, where appropriate, for providing material deemed essential to the claimed subject matter by an examining authority or court.

Modifications may be made to the foregoing without departing from the basic aspects of the invention. Although the invention has been described in substantial detail with reference to one or more specific embodiments, those of ordinary skill in the art will recognize that changes may be made to the embodiments specifically disclosed in this application, and yet these modifications and improvements are within the scope and spirit of the invention. The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. Thus, the terms and expressions which have been employed are used as terms of description and not of limitation, equivalents of the features shown and described, or portions thereof, are not excluded, and it is recognized that various modifications are possible within the scope of the invention. Embodiments of the invention are set forth in the following claims.

Various features of the embodiments of the invention disclosed herein are, for brevity, described in the context of a single embodiment, but may also be provided separately or in any suitable sub-combination. All combinations of the embodiments are specifically embraced by the illustrative embodiments disclosed herein just as if each and every combination was individually and explicitly disclosed. In addition, all sub-combinations listed in the embodiments describing such variables are also specifically embraced by the present compositions and are disclosed herein just as if each and every such sub-combination was individually and explicitly disclosed herein.

The invention will be further described with reference to the examples described herein; however, it is to be understood that the invention is not limited to such examples.

### EXAMPLES

### Example 1: Exemplary Transdermal Devices to Treat Long COVID

This example describes use of transdermal devices as provided herein for treating or ameliorating so-called “long COVID” or Post-COVID Conditions (PCC), or post-acute sequelae of SARS COV-2 infection (PASC).

Several patients present to the clinic with various symptoms including: difficulty thinking or concentrating (sometimes referred to as “brain fog”); headache; sleep problems; dizziness or lightheadedness standing up; pins-and-needles feelings; change in or loss of smell or taste; and/or depression or anxiety. Medical histories include acute COVID infections.

Each patient is administered a transdermal device (apply to the arm, chest, stomach, upper back or lower back) manufactured to deliver ZIVERDOX™, or the therapeutic drug combination zinc, ivermectin, and doxycycline.

The transdermal device has three separate compartments, each compartment containing one of zinc, ivermectin and doxycycline, each formulated in a higher dosage (ivermectin 48 mg, doxycycline 400 mg and zinc chelate 100 mg) than would be formulated of the same drug combination was to be orally administered (which would be ivermectin 12 mg, doxycycline 100 mg and zinc chelate 25 mg).

The zinc, ivermectin and doxycycline are separately formulated in DMSO, and contained in separate compartments in the transdermal device.

Each patient is given between 7 and 10 transdermal devices to self-apply once a day.

The surface of the transdermal delivery device to be applied to the skin has an (one) outer surface, layer or covering or laminate (a “release liner”) that is removable by the patient just before being place against the skin.

Removal of the release liner also exposes adhesive on the transdermal device that both retains the transdermal device on the skin without causing irritation, and keeps the drugs being transdermally delivered from “leaking out” of the transdermal device. The adhesive can comprise an acrylate polymer or any pressure-sensitive adhesive (PSA).

### Example 2: Exemplary Transdermal Devices to Prevent Severe Clinical COVID

This example describes use of transdermal devices as provided herein for preventing severe COVID infection.

Individuals who expect to be exposed to a potentially infectious environment such as an airplane or subway trip are administered a transdermal device (apply to the arm, chest, stomach, upper back or lower back) manufactured to deliver ZIVERDOX™, or the therapeutic drug combination zinc, ivermectin, and doxycycline.

The transdermal device has three separate compartments, each compartment containing one of zinc, ivermectin and doxycycline, each formulated in a higher dosage (ivermectin 48 mg, doxycycline 400 mg and zinc chelate 100 mg) than would be formulated of the same drug combination was to be orally administered (which would be ivermectin 12 mg, doxycycline 100 mg and zinc chelate 25 mg).

The zinc, ivermectin and doxycycline are separately formulated in DMSO, and contained in separate compartments in the transdermal device.

These transdermal devices can be self-applied before the individual's exposure, but no more than once a day.

The surface of the transdermal delivery device to be applied to the skin has an (one) outer surface, layer or covering or laminate (a “release liner”) that is removable by the patient just before being place against the skin.

Removal of the release liner also exposes adhesive on the transdermal device that both retains the transdermal device on the skin without causing irritation, and keeps the drugs being transdermally delivered from “leaking out” of the transdermal device. The adhesive can comprise an acrylate polymer or any pressure-sensitive adhesive (PSA).

### Example 3

A 64-year-old patient with diabetes and hypertension had acquired COVID as proven by PCR. He had a sore throat and cough and his oxygen tension by oximetry fell to 82. He was treated with a transdermal patch containing higher dose of active antiviral drugs including 48 mg of ivermectin, 300 mg of doxycycline, and 50 mg of zinc chelate. The transdermal patch had three separate compartments, each compartment containing one of ivermectin, doxycycline and zinc chelate. Each active ingredient was formulated with a gelling agent and water. The patch remained adhered to the left side of the chest for 3 days. Blood levels of each of the ivermectin, doxycycline and zinc chelate rose to a peak at 10 hrs, The patient's cough continued but slowly resolved and he was not breathless. The area under the curve for each of the ivermectin, doxycycline and zinc chelate was greater than that of single oral medications and it persisted for 6 days compared with 48 hr in orally treated patients. The patient oxygen saturation improved within 2 days with oximetry rising to 96%.

This example led to the preventative use of such a patch. In 3 patients none acquired Covid-19 when cohabiting with a Covid-19 infected person.

A number of embodiments of the invention have been described. Nevertheless, it can be understood that various modifications may be made without departing from the spirit and scope of the invention. Accordingly, other embodiments are within the scope of the following claims.

1. A pharmaceutical composition or a therapeutic combination of drugs, comprising:
(a) (i) metformin (or GLUCOPHAGE™) and/or
(ii) a 3C-like (3CLpro) protease (also known as main protease (Mpro), also known as C30 endopeptidase or 3-chymotrypsin-like protease) inhibitor, optionally ensitrelvir (or XOCOVA™), or ensitrelvir fumaric acid,
wherein optionally the metformin is formulated or dosaged at between about 100 mg to mg per unit dosage (optionally, per tablet); or the metformin is formulated or dosage at between about 0.1 ml to 25 ml liquid metformin/day; and,
wherein optionally the ensitrelvir or ensitrelvir fumaric acid is formulated or dosaged at between about 100 mg to 500 mg per unit dosage, or at about 100 mg, 125 mg or 150 mg per unit dosage (optionally, per tablet); and
(b) two of any of the following drugs or drug combinations if only metformin or the 3CLpro inhibitor are included, or one of (or at least one of) any of the following drugs or drug combinations if both metformin and 3CLpro are included:
(i) an avermectin class drug, or ivermectin (or STROMECTOL™);
(ii) an avermectin class drug, or ivermectin and an antibiotic (optionally doxycycline or azithromycin);
(iii) an avermectin class drug, or ivermectin; an antibiotic (optionally doxycycline or azithromycin), and zinc, and optionally also vitamin D or cholecalciferol, optionally with or without vitamin C;
(iv) an antibiotic;
(v) ivermectin; and, an antibiotic (optionally doxycycline or azithromycin), at least one vitamin and zinc;
(vi) opaganib or YELIVA™;
(vii) opaganib and: an avermectin class drug, or ivermectin; an antibiotic (optionally doxycycline or azithromycin), and zinc, and optionally also vitamin D or cholecalciferol;
(viii) plitidepsin (also known as dehydrodidemnin B), or APLIDIN™;
(ix) plitidepsin and: an avermectin class drug, or ivermectin; an antibiotic (optionally doxycycline or azithromycin), and zinc, and optionally also vitamin D or cholecalciferol, and optionally also vitamin D or cholecalciferol;
(x) lopinavir, ritonavir (or NORVIR™) and/or oseltamivir,
(xi) lopinavir, ritonavir and/or oseltamivir, and: an avermectin class drug, or ivermectin; an antibiotic (optionally doxycycline or azithromycin), and zinc, and optionally also vitamin D or cholecalciferol;
(xii) nirmatrelvir and ritonavir (or PAXLOVID™),
(xiii) nirmatrelvir and ritonavir (or PAXLOVID™), and: an avermectin class drug, or ivermectin; an antibiotic (optionally doxycycline or azithromycin), and zinc, and optionally also vitamin D or cholecalciferol;
(xiv) chloroquine (optionally, ARALEN™), chloroquine phosphate, chloroquine diphosphate, hydroxychloroquine (optionally, PLAQUENIL™);
(xv) chloroquine, chloroquine phosphate, chloroquine diphosphate, hydroxychloroquine; and: an avermectin class drug, or ivermectin; an antibiotic (optionally doxycycline or azithromycin), and zinc, and optionally also vitamin D or cholecalciferol;
(xvi) abacavir, acyclovir optionally, (ACICLOVIR™), adefovir, amantadine, ampligen, amprenavir (optionally, AGENERASE™), aprepitant, arbidol, atazanavir, atripla, balavir, baloxavir marboxil (XOFLUZA™), bepotastine, bevirimat, bictegravir, biktarvy, brilacidin, cidofovir, caspofungin, lamivudine and zidovudine (optionally, COMBVIR™), cobicstat, colisitin, cocaine, delavirdine, descovy, didanosine, docosanol, dolutegravir, ecoliever, edoxudine, elvitegravir, enfuvirtide, entecavir, epirubicin, epoprostenol, etravirine, famciclovir, fomivirsen, fosamprenavi, foscarnet, fosfonet, galidesivir, ibacitabine, icatibant, idoxuridine, ifenprodil, imiquimod, imunovir, indinavir, inosine, an interferon (optionally interferon type I, interferon type II and/or interferon type III), lamivudine, loviride, ledipasvir, leronlimab, maraviroc, methisazone, moroxydine, nexavir, norvir, a nucleoside analogue (optionally brincidofovir, didanosine, favipiravir (also known as T-705, avigan, or favilavir, Toyama Chemical, Fujifilm, Japan), vidarabine, galidesivir (optionally, BCX4430, IMMUCILLIN-A™), cytarabine, gemcitabine, lamivudine, zalcitabine, abacavir, acyclovir, entecavir, stavudine, telbivudine, zidovudine, idoxuridine and/or trifluridine or any combination thereof), oseltamivir (or TAMIFLU™), peginterferon alfa-2a, penciclovir, peramivir (optionally, RAPIVAB™), perfenazine, pleconaril, plurifloxacin, podophyllotoxin, pyramidine, raltegravir, rifampicin, rilpivirine, rimantadine, ritonavir, saquinavir, sofosbuvir, stavudine, telaprevir, tegobuv, tipranavir, trifluridine, trizivir, tromantadine, truvada, valaciclovir (optionally, VALTREX™), valganciclovir, valrubicin, vapreotide, vicriviroc, vidarabine, viramidine, velpatasvir, vivecon, zalcitabine, zanamivir (optionally, RELENZA™), zidovudine, or any combination thereof;
(xvii) any one or more drug or drugs in (xiv); and: an avermectin class drug, or ivermectin; an antibiotic (optionally doxycycline or azithromycin), and zinc, and optionally also vitamin D or cholecalciferol;
(xviii) molnupiravir, emtricitabine (or EMTRIVA™), efavirenz (or SUSTIVA™) or tenofovir; or molnupiravir with emtricitabine and tenofovir (or ATRIPLA™);
(xix) molnupiravir, emtricitabine, efavirenz or tenofovir, and: an avermectin class drug, or ivermectin; an antibiotic (optionally doxycycline or azithromycin), and zinc, and optionally also vitamin D or cholecalciferol;
(xx) zanamivir (or RELENZA™);
(xxi) zanamivir, and: an avermectin class drug, or ivermectin; an antibiotic (optionally doxycycline or azithromycin), and zinc, and optionally also vitamin D or cholecalciferol;
(xxii) efavirenz (optionally SUSTIVA™);
(xxiii) efavirenz, and: an avermectin class drug, or ivermectin; an antibiotic (optionally doxycycline or azithromycin), and zinc, and optionally also vitamin D or cholecalciferol;
(xxiv) nelfinavir (VIRACEPT™) or oseltamivir (or TAMIFLU™),
(xxv) nelfinavir (VIRACEPT™) or oseltamivir (or TAMIFLU™), and: an avermectin class drug, or ivermectin; an antibiotic (optionally doxycycline or azithromycin), and zinc, and optionally also vitamin D or cholecalciferol;
(xxvi) a thiazolide class drug, optionally nitazoxanide (optionally ALINIA™, or NIZONIDE™) or tizoxanide (or 2-Hydroxy-N-(5-nitro-2-thiazolyl)benzamide);
(xxvii) a thiazolide class drug, optionally nitazoxanide, and: an avermectin class drug, or ivermectin; an antibiotic (optionally doxycycline or azithromycin), and zinc, and optionally also vitamin D or cholecalciferol;
(xxviii) remdesivir (optionally, GS-5734™, Gilead Sciences, or VEKLURY™);
(xxix) molnupiravir (or LAGEVRIO™);
(xxx) molnupiravir, and: an avermectin class drug, or ivermectin; an antibiotic (optionally doxycycline or azithromycin), and zinc, and optionally also vitamin D or cholecalciferol;
(xxxi) nevirapine (or VIRAMUNE™);
(xxxii) nevirapine (or VIRAMUNE™), and: an avermectin class drug, or ivermectin; an antibiotic (optionally doxycycline or azithromycin), and zinc, and optionally also vitamin D or cholecalciferol;
(xxxiii) tenofovir alafenamide, tenofovir disoproxil or tenofovir;
(xxxiv) tenofovir alafenamide, tenofovir disoproxil or tenofovir, and: an avermectin class drug, or ivermectin; an antibiotic (optionally doxycycline or azithromycin), and zinc, and optionally also vitamin D or cholecalciferol;
(xxxv) salamectin (or SELEHOLD™, REVOLT™, SELARID™ or SENERGY™);
(xxxvi) salamectin, and: an avermectin class drug, or ivermectin; an antibiotic (optionally doxycycline or azithromycin), and zinc, and optionally also vitamin D or cholecalciferol;
(xxxvii) an inhibitor of an S-phase kinase-associated protein 2 (SKP2), or dioscin, or niclosamide, or NICLOCIDE™, FENASAL™, or PHENASAL™,
(xxxviii) an inhibitor of an S-phase kinase-associated protein 2 (SKP2), or dioscin, or niclosamide, and: an avermectin class drug, or ivermectin; an antibiotic (optionally doxycycline or azithromycin), and zinc, and optionally also vitamin D or cholecalciferol;
(xxxix) ribavirin or tribavirin (or COPEGUS™, REBETOL™, or VIRAZOLE™);
(xxxx) ribavirin or tribavirin, and: an avermectin class drug, or ivermectin; an antibiotic (optionally doxycycline or azithromycin), and zinc, and optionally also vitamin D or cholecalciferol;
(xxxxi) a viral, or a coronavirus or a COVID-19, protease inhibitor, optionally ASC09 (CAS registry no. 1000287-05-7) (Janssen Research and Development, LLC), ritonavir or ASC09 and ritonavir, or a JAK1/2 inhibitor (optionally baricitinib), optionally compound 11r (University of Lubeck, Germany, see optionally, Zhang et al J. Med Chem 2020, Feb. 11, 2020), or darunavir (or PREZISTA™), cobicistat (or TYBOST™) or darunavir and cobicistat;
(xxxxii) a viral, or a coronavirus or a COVID-19, protease inhibitor, ritonavir, a JAK1/2 inhibitor (optionally baricitinib), or darunavir (or PREZISTA™) or cobicistat, and: an avermectin class drug, or ivermectin; an antibiotic (optionally doxycycline or azithromycin), and zinc, and optionally also vitamin D or cholecalciferol;
(xxxxiii) an mucolytic therapy or drug, optionally acetylcysteine, ambroxol, bromhexine, carbocisteine, erdosteine, mecysteine or dornase alfa, or an expectorant, optionally guaifenesin;
(xxxxiv) an mucolytic therapy or drug, and: an avermectin class drug, or ivermectin; an antibiotic (optionally doxycycline or azithromycin), and zinc, and optionally also vitamin D or cholecalciferol;
(xxxxv) an angiotensin-converting enzyme 2 (ACE2) inhibitor, optionally captopril, enalapril, lisinopril, benazepril, fosinopril, quinapril, ramipril, perindopril, moexipril or trandolapril;
(xxxxvi) an angiotensin-converting enzyme 2 (ACE2) inhibitor, and: an avermectin class drug, or ivermectin; an antibiotic (optionally doxycycline or azithromycin), and zinc, and optionally also vitamin D or cholecalciferol;
(xxxxvii) an anti-vascular endothelial growth factor (VEGF) (optionally VEGF-A) drug or antibody, optionally bevacizumab (or AVASTIN™);
(xxxxviii) an anti-vascular endothelial growth factor (VEGF) drug or antibody or bevacizumab and: an avermectin class drug, or ivermectin; an antibiotic (optionally doxycycline or azithromycin), and zinc, and optionally also vitamin D or cholecalciferol;
(xxxxvix) a protease inhibitor, optionally danoprevir, optionally a serine protease inhibitor, optionally camostat (or FOIPAN™) or narlaprevir (optionally ARLANSA™)
(xxxxx) a protease inhibitor, danoprevir, a serine protease inhibitor, camostat or narlaprevir, and: an avermectin class drug, or ivermectin; an antibiotic (optionally doxycycline or azithromycin), and zinc, and optionally also vitamin D or cholecalciferol;
(xxxxxi) anti-PD-1 checkpoint inhibitor, optionally camrelizumab, or a PD-1 inhibitor (or cemiplimab, dostarlimab, pembrolizumab or nivolumab) or a PD-L1 inhibitor (or atezolizumab, avelumab, cosibelimab or durvalumab);
(xxxxxii) anti-PD-1 checkpoint inhibitor, or camrelizumab, or a PD-1 inhibitor or a PD-L1 inhibitor, and: an avermectin class drug, or ivermectin; an antibiotic (optionally doxycycline or azithromycin), and zinc, and optionally also vitamin D or cholecalciferol;
(xxxxxiii) thalidomide, or thalidomide and glucocorticoid (optionally low-dose glucocorticoid), or and thalidomide and celecoxib;
(xxxxxiv) thalidomide, or thalidomide and glucocorticoid (optionally low-dose glucocorticoid), and: an avermectin class drug, or ivermectin; an antibiotic (optionally doxycycline or azithromycin), and zinc, and optionally also vitamin D or cholecalciferol;
(xxxxxV) a compound or antibody capable of binding complement factor C5 and blocking membrane attack complex formation, or eculizumab;
(xxxxxvi) a compound or antibody capable of binding complement factor C5 and blocking membrane attack complex formation, or eculizumab, and: an avermectin class drug, or ivermectin; an antibiotic (optionally doxycycline or azithromycin), and zinc, and optionally also vitamin D or cholecalciferol;
(xxxxxvii) a cathepsin inhibitor, optionally a cathepsin K, B or L inhibitor, or relacatib or balicatib;
(xxxxxviii) a cathepsin inhibitor, or relacatib or balicatib, and: an avermectin class drug, or ivermectin; an antibiotic (optionally doxycycline or azithromycin), and zinc, and optionally also vitamin D or cholecalciferol;
(xxxxxix) a synthetic nucleoside analog or derivative, or N4-hydroxycytidine, or a prodrug of N4-hydroxycytidine, optionally molnuvpiravir (Merck), or favipiravir (also known as T-705 or AVIGAN™), or favilavir, Toyama Chemical, Fujifilm, Japan, or FABIFLU™ (Glenmark Pharmaceuticals), optionally dosaged at 800 mg bid;
(xxxxxx) a synthetic nucleoside analog or derivative, and: an avermectin class drug, or ivermectin; an antibiotic (optionally doxycycline or azithromycin), and zinc, and optionally also vitamin D or cholecalciferol,
wherein the synthetic nucleoside analog or derivative, or N4-hydroxycytidine, or the prodrug of N4-hydroxycytidine, optionally molnuvpiravir or favipiravir, is given as between about 10 mg to 3 gm per dose, or between about 10 mg to 3 gm per day, or can be dosed either as a single dose or given one, two, three or four times a day, or is administered at 200 to 800 mg twice daily, or 200, 400, 600 or 800 mg twice daily, or at 200 to 800 mg three times a day, or at 200, 400, 600 or 800 mg three times a day, or is administered at 200 to 800 mg three times a day for between about 2 to 15 days, or for about 2, 3, 4, 5, 6, 7, 8, 9, 10, 11 or 12 days, and optionally when combined with other drugs a lower dosage is used, optionally administered at 100 or 200 mg three times a day for between about 5 to 15 days, or for about 7, 8, 9, 10, 11 or 12 days;
(xxxxxxi) an anti-androgen drug, and optionally the anti-androgen drug is bicalutamide, or CASODEX™, or dutasteride (or AVODART™), and optionally the anti-androgen drug comprises a 5α-reductase inhibitor, and optionally the 5α-reductase inhibitor comprises finasteride (or PROSCAR™, PROPECIA™, or FINIDE™);
(xxxxxxii) an anti-androgen drug, and optionally the anti-androgen drug is bicalutamide, or CASODEX™, or dutasteride (or AVODART™), and: an avermectin class drug, or ivermectin; an antibiotic (optionally doxycycline or azithromycin), and zinc, and optionally also vitamin D or cholecalciferol;
(xxxxxxiii) an anti-malarial drug, wherein optionally the anti-malarial drug comprises mefloquine (or LARIAM™, MEPHAQUIN™, or MEFLIAM™), wherein optionally the mefloquine is formulated for oral administration, optionally in tablet or capsule form, optionally as 200 mg, 250 mg or 300 mg tablets;
(xxxxxxiv) an anti-malarial drug, and: an avermectin class drug, or ivermectin; an antibiotic (optionally doxycycline or azithromycin), and zinc, and optionally also vitamin D or cholecalciferol;
(xxxxxxv) a peroxisome proliferator-activated receptor (PPAR) agonist, wherein optionally the PPAR agonist comprises fenofibrate, or TRICOR™, FENOBRAT™, FENOGLIDE™ or LIPOFEN™, optionally the PPAR agonist comprises a combination of fenofibrate and pravastatin, or PRAVAFENIX™, or the PPAR agonist comprises bezafibrate, or BEZALIP™, or combination of bezafibrate and chenodeoxycholic acid, or HEPACONDA™, or aluminium clofibrate, or alfibrate, or ciprofibrate, or clinofibrate or LIPOCLIN™, or clofibrate or ATROMID-S™, or clofibride, or gemfibrozil or LOPID™, or ronifibrate, or simfibrate or CHOLESOLVIN™, or any combination thereof,
(xxxxxxvi) a peroxisome proliferator-activated receptor (PPAR) agonist, and: an avermectin class drug, or ivermectin; an antibiotic (optionally doxycycline or azithromycin), and zinc, and optionally also vitamin D or cholecalciferol;
(xxxxxxvii) an acetaldehyde dehydrogenase inhibitor, optionally disulfiram, or ANTABUS™, or ANTABUSE™, optionally formulated as an extended, sustained or slow-release disulfiram formulation, optionally the extended, sustained or slow-release disulfiram is formulated as a tablet, a capsule or in an injectable, amphiphilic, absorbable, depot-forming drug delivery system (DDS),
(xxxxxxviii) an acetaldehyde dehydrogenase inhibitor, and: an avermectin class drug, or ivermectin; an antibiotic (optionally doxycycline or azithromycin), and zinc, and optionally also vitamin D or cholecalciferol;
(xxxxxxix) a nicotinic antagonist, a dopamine agonist or a noncompetitive N-Methyl-D-aspartate (NMDA) antagonist, optionally amantadine, or GOCOVRI™, or SYMADINE™, or SYMMETREL™, optionally dosaged at between about 100 to 200 mg per dose, optionally formulated as tablets or capsules,
(xxxxxxx) a nicotinic antagonist, a dopamine agonist or a noncompetitive N-Methyl-D-aspartate (NMDA) antagonist, and: an avermectin class drug, or ivermectin; an antibiotic (optionally doxycycline or azithromycin), and zinc, and optionally also vitamin D or cholecalciferol;
(xxxxxxxi) a mitochondrial sensitizer, optionally proguanil or chlorguanide (or PALUDRINE™), or a malarial cytochrome bc1 complex inhibitor, optionally atovaquone (or MEPRON™), or a combination of proguanil and atovaquone (or MALARONE™),
and optionally the proguanil, atovaquone or the combination of proguanil and atovaquone are formulated for oral administration, optionally as tablets, optionally the unit dosage of atovaquone is 250 mg, 300 mg, 350 mg, 400 mg, 500 mg or 1 gram, and the unit dosage of proguanil is 100 mg, 250 mg, 300 mg, 350 mg or 400 mg;
(xxxxxxxii) a mitochondrial sensitizer or a malarial cytochrome bc1 complex inhibitor, and: an avermectin class drug, or ivermectin; an antibiotic (optionally doxycycline or azithromycin), and zinc, and optionally also vitamin D or cholecalciferol;
(xxxxxxxiii) a dendrimer, optionally astodrimer sodium (Starpharma, Melbourne, Australia);
(xxxxxxxiv) a dendrimer, and: an avermectin class drug, or ivermectin; an antibiotic (optionally doxycycline or azithromycin), and zinc, and optionally also vitamin D or cholecalciferol;
(xxxxxxxv) an antihistamine class drug, optionally azelastine, or ASTELIN™, OPTIVAR™, ALLERGODIL™, bepotastine (or TALION™, BEPREVE™), brompheniramine, fexofenadine or ALLEGRA™, pheniramine or AVIL™, or chlorpheniramine;
(xxxxxxxvi) an antihistamine class drug, and: an avermectin class drug, or ivermectin; an antibiotic (optionally doxycycline or azithromycin), and zinc, and optionally also vitamin D or cholecalciferol;
(xxxxxxxvii) a selective serotonin reuptake inhibitor (SSRI) class drug, optionally fluvoxamine, or LUVOX™, FAVERIN™, FLUVOXIN™; a peroxisome proliferator-activated receptor (PPAR) agonist, wherein optionally the PPAR agonist comprises fenofibrate, or TRICOR™, FENOBRAT™, FENOGLIDE™ or LIPOFEN™, optionally the PPAR agonist comprises a combination of fenofibrate and pravastatin, or PRAVAFENIX™, or the PPAR agonist comprises bezafibrate, or BEZALIP™, or combination of bezafibrate and chenodeoxycholic acid, or HEPACONDA™, or aluminium clofibrate, or alfibrate, or ciprofibrate, or clinofibrate or LIPOCLIN™, or clofibrate or ATROMID-S™, or clofibride, or gemfibrozil or LOPID™, or ronifibrate, or simfibrate or CHOLESOLVIN™, or any combination thereof;
(xxxxxxxviii) a selective serotonin reuptake inhibitor (SSRI) class drug, and: an avermectin class drug, or ivermectin; an antibiotic (optionally doxycycline or azithromycin), and zinc, and optionally also vitamin D or cholecalciferol;
(xxxxxxxix) clofazimine, or LAMPENE™, optionally dosaged at about 100 mg per day, or between about 50 mg and 150 mg per day, and/or colchicine (or COLCRYS™, MITIGARE™), optionally dosaged at about 0.1 mg to 5 mg per day;
(xxxxxxxx) clofazimine or colchicine, and: an avermectin class drug, or ivermectin; an antibiotic (optionally doxycycline or azithromycin), and zinc, and optionally also vitamin D or cholecalciferol;
(xxxxxxxxi) a selective estrogen receptor modulator (SERM), or toremifene (or FARESTON™), or clomifene or clomiphene (or CLOMID™, SEROPHENE™);
(xxxxxxxxii) a selective estrogen receptor modulator (SERM), or toremifene or clomifene or clomiphene, and: an avermectin class drug, or ivermectin; an antibiotic (optionally doxycycline or azithromycin), and zinc, and optionally also vitamin D or cholecalciferol;
(xxxxxxxxiii) an alpha-ketoamide (a-ketoamide), wherein optionally the alpha-ketoamide is a structure as described by Zhang et al, J. Med. Chem. 2020, 63, 9, 4562-4578, or Meng et al Chem. Sci. (2019) vol. 10, pg 5156 (optionally the structure KAM-2);
(xxxxxxxxiv) an alpha-ketoamide, and: an avermectin class drug, or ivermectin; an antibiotic (optionally doxycycline or azithromycin), and zinc, and optionally also vitamin D or cholecalciferol;
(xxxxxxxxv) an acylsulfonamide inhibitor of the NS3-4A serine protease, or paritaprevir, or ombitasvir, or paritaprevir and ombitasvir with ritonavir (TECHNIVIE™);
(xxxxxxxxvi) paritaprevir (or VERUPREVIR™), or ombitasvir, or paritaprevir and ombitasvir with ritonavir, and: an avermectin class drug, or ivermectin; an antibiotic (optionally doxycycline or azithromycin), and zinc, and optionally also vitamin D or cholecalciferol;
(xxxxxxxxvii) at least one vitamin, and optionally the at least one vitamin comprises: vitamin B3 (or pyridine-3-carboxylic acid, niacin or nicotinic acid, or vitamin B3 or niacin administered as a slow release form (or NIASPAN FCT™), vitamin D (optionally D2, or ergocalciferol), or Vitamin D3 or cholecalciferol, optionally administered at about 1000 to 4000 ugm/day; vitamin B12, vitamin B6 (or pyridoxine); vitamin K; vitamin A; vitamin E; and/or, vitamin C (optionally administered at 500 mg bid);
(xxxxxxxxviii) at least one vitamin, and: an avermectin class drug, or ivermectin; an antibiotic (optionally doxycycline or azithromycin), and zinc;
(xxxxxxxxix) a compound, drug or formulation that decreases stomach acid production or decreases stomach pH, wherein optionally the compound, drug or formulation comprises famotidine, or PEPCID™, and optionally the famotidine is administered at a dosage of between about 10 to 60 mg per day, or between about 20 to 40 mg per day;
(xxxxxxxxx) famotidine, and: an avermectin class drug, or ivermectin; an antibiotic (optionally doxycycline or azithromycin), and zinc;
(xxxxxxxxxi) an immunosuppressive drug, wherein optionally the immunosuppressive drug comprises tocilizumab or atlizumab, or ACTEMRA™, or ROACTEMRA™, or a calcineurin inhibitor (CNI), wherein the CNI comprises ciclosporin (or cyclosporine or cyclosporin), or NEORAL™, or SANDIMMUNE™, or tacrolimus, or PROTOPIC™, or PROGRAF™.
(xxxxxxxxxii) an immunosuppressive drug or a calcineurin inhibitor, and: an avermectin class drug, or ivermectin; an antibiotic (optionally doxycycline or azithromycin), and zinc;
(xxxxxxxxxiii) an anti-inflammatory therapy or at least one anti-inflammatory therapy drug, wherein optionally the anti-inflammatory therapy or drug comprises: a sphingosine kinase-2 (SK2) selective inhibitor (optionally, opaganib (optionally, YELIVA™), sirolimus, a JAK1/2/TYK2 inhibitor (optionally ruxolitinib), an anti-CD47 mAb (optionally meplazumab), a cyclooxygenase (COX) (optionally, COX2) inhibitor, a glucocorticoid (optionally a synthetic glucocorticoid, hydrocortisone, dexamethasone (or DEXTENZA™, OZURDEX™, or NEOFORDEX™) or cortisol, or CORTEF™), plitidepsin or dehydrodidemnin B, or APLIDIN™; and/or
(xxxxxxxxxiv) an anti-inflammatory therapy or at least one anti-inflammatory therapy drug, and: an avermectin class drug, or ivermectin; an antibiotic (optionally doxycycline or azithromycin), and zinc;
wherein optionally the avermectin class drug comprises: ivermectin (or STROMECTOL™), moxidectin (or CYDECTIN™, EQUEST™, QUEST™), selamectin (or STRONGHOLD™), a milbemycin (optionally milbemectin, milbemycin oxime, moxidectin or nemadectin), doramectin (or DECTOMAX™), eprinomectin or abamectin, and optionally the avermectin class drug or ivermectin is dosaged for administration at between about 50 to 2000 μgm/kg body weight per day, optionally dosaged at 200 μgm/kg per day, or about 14 mg/day, optionally dosaged at 220, 225, 150, 275 or 300 μgm/kg per day, or optionally dosaged at 12, 14, 16, 18, 20, 22, 24 or 26 mg per day, or optionally dosaged at between about 10 to 30 mg per day,
and optionally the avermectin class drug or ivermectin is dosaged for administration at between about 5 to 480 or between about 3 to 500 mg per day,
and optionally the avermectin class drug (optionally ivermectin) is formulated or dosaged at between about 15 to 150 mg/kg, or is about 18, 24, 30, 35, 40, 35, 50, 55, 60, 65, 70, 75, 80, 85, 90, 95, 100, 110 or 120 or more mg/kg,
and optionally the antibiotic comprises a tetracycline class drug, and optionally the tetracycline class drug comprises tetracycline, doxycycline, chlortetracycline, oxytetracycline, glycylcycline, fluorocycline or demeclocycline, tetracycline or SUMYCIN™; chlortetracycline or AUREOMYCIN™; oxytetracycline; demeclocycline or DECLOMYCIN™ DECLOSTATIN™, LEDERMYCIN™, BIOTERCICLIN™, DEGANOL™, DETECLO™, DETRAVIS™, MECICLIN™, MEXOCINE™, CLORTETRIN™; lymecycline; meclocycline;
metacycline; minocycline or MINOCIN™; rolitetracycline; doxycycline, or DORYX™, DOXYHEXA™, DOXYLIN™; tigecycline or TYGACIL™; eravacycline or XERAVA™; sarecycline or SEYSARA™; omadacycline or NUZYRA™; or any combination thereof,
and optionally the therapeutic combination comprises about 25 mg to about 600 mg of the tetracycline class drug, or tetracycline, doxycycline, chlortetracycline, oxytetracycline or demeclocycline,
and optionally the antibiotic comprises a macrolide drug, and optionally the macrolide drug comprises azithromycin, optionally dosaged at between about 50 mg to about 2000 mg per dose or per day (optionally, ZITHROMAX™, or AZITHROCIN™, optionally an oral extended- or delayed-release formulation of azithromycin, or ZMAX™), clarithromycin (optionally, BIAXIN™), erythromycin (optionally, ERYTHROCIN™), or fidaxomicin (optionally, DIFICID™ or DIFICLIR™), troleandomycin (optionally, TEKMISIN™), tylosin (optionally, TYLOCINE™ or TYLAN™), solithromycin (optionally, SOLITHERA™), oleandomycin (or SIGMAMYCINE™), midecamycin, roxithromycin, kitasamycin or turimycin, josamycin, carbomycin or magnamycin, and/or spiramycin,
and optionally the antibiotic comprises azithromycin, and optionally the therapeutic combination comprises between about 50 mg to about 2000 mg azithromycin, or optionally the azithromycin comprises an oral extended-release formulation of azithromycin,
and optionally the antibiotic comprises a nitroimidazole, or a 2-, 4- and/or 5-nitroimidazole, or a 5-nitro nitroimidazole comprising metronidazole, tinidazole, nimorazole, dimetridazole, pretomanid, ornidazole, megazol and/or azanidazole, or a 2-nitromidazoles comprising benznidazole,
and optionally the antibiotic comprises a-lactam antibiotic such as penicillin class drug or a cephalosporin class drug,
and optionally the cephalosporin class drug comprises cefalexin or cephalexin, cefadroxil or cefadroxyl, cefazolin or cephazolin, cefapirin or cephapirin, cefalotin or cephalothn, cefradine or corcephradine, cefprozil or cefproxil, cefuroxime, ceftolozane, cefonicid or cefaclor,
and optionally the penicillin class drug comprises penicillin V, penicillin G, cloxacillin, dicloxacillin, flucloxacillin, methicillin, nafcillin, oxacillin, ampicillin, amoxicillin, pivampicillin, bacampicillin, metampicillin, talampicillin, hetacillin, carbenicillin, ticarcillin, temocillin, mezlocillin, piperacillin, azlocillin, clavulanic acid, sulbactam or tazobactam,
and optionally the antibiotic comprises a quinolone class antibiotic such as a fluoroquinolone, or comprises ciprofloxacin, garenoxacin, gatifloxacin, gemifloxacin, levofloxacin or moxifloxacin,
and optionally the antibiotic comprises an ansamycin class drug, or rifampicin, also known as rifampin, bedaquiline,
and optionally the antibiotic comprises artemisinin, amodiaquine, proguanil, sulfadoxine, sulfamethoxypyridazine, pyrimethamine, linezolid, quinine, quinidine, cinchonine, cinchonidine (or quinimax), hydroxychloroquine or chloroquine,
and optionally the at least one vitamin comprises vitamin D or cholecalciferol, or vitamin C, or vitamin D or cholecalciferol and vitamin C, and vitamin D or cholecalciferol is dosaged at between about 3,000 to about 100,000 units vitamin D or cholecalciferol, and optionally the therapeutic combination comprises between about 10,000 to about 50,000 units vitamin D or cholecalciferol, and optionally the vitamin comprises vitamin C, and optionally the vitamin C is formulated or administered at a dosage of between about 500 to 5000 units (U) per dose,
and optionally the zinc comprises or is formulated as: a zinc salt, a zinc chelate, a zinc sulphate, a zinc acetate, a zinc gluconate or a zinc picolinate, and optionally the therapeutic combination comprises between about 1 mg to 250 mg zinc, zinc sulphate, zinc acetate, zinc gluconate or zinc picolinate, or zinc oxide nanoparticles, optionally at a dosage of between about 1 mg to 250 mg,
and optionally the pharmaceutical composition or a therapeutic combination further comprises copper, optionally administered or formulated at a dosage of between about 1 to 200 mg per day, wherein optionally the copper is administered or formulated as cupric chloride and administered intravenously formulated at about 0.4 mg/ml;
and optionally the pharmaceutical composition or a therapeutic combination further comprises selenium, optionally administered as selenious acid formulated at about 65.4 mcg/ml (or μ/ml), and optionally the selenium is administered at a dosage of between about 50 to 100 μ/ml, optionally between about 60 to 100 μgm per day is administered to an adult, and only up to 60 μgm per day for pediatric patients.

2. A product of manufacture comprising a pharmaceutical composition or a therapeutic combination of drugs of claim 1,
wherein optionally the product of manufacture comprises a pharmaceutical formulation, or a dressing or a dermal patch, or an implant,
and optionally the pharmaceutical composition or a therapeutic combination is formulated as: a gel, a liquid, an aerosol, a spray, a mist, a lotion, a cream, a powder, a tablet, a capsule, a pill, a geltab, an enema, a suppository, a transdermal or intraoral patch, or an injectable formulation, a subcutaneous, an intramuscular (IM) or intravenous (IV) formulation, to make a pharmaceutical formulation.

3. A kit comprising a pharmaceutical composition or a therapeutic combination of drugs of claim 1, or a product of manufacture of claim 2, optionally to deliver both oral and transdermal medications as needed.

4. Use of a pharmaceutical composition or a therapeutic combination of drugs of claim 1, or a product of manufacture of claim 2, or a kit of claim 3, for treating, preventing or ameliorating (optionally decreasing the symptoms of, or decreasing the mortality of) an infection caused by a virus, optionally a virus in the subfamily Orthocoronavirinae, or a virus in the family Coronaviridae, Arteriviridae, Roniviridae and/or Mesoniviridae, or a virus in the subfamily subfamily Letovirinae and Orthocoronavirinae; or a virus in the order Nidovirales, or an Alphacoronavirus, betacoronavirus, gammacoronavirus or deltacoronavirus, or a severe acute respiratory syndrome coronavirus 2(SARS-COV-2) virus or variant thereof, and optionally the SARS-COV-2 is an omicron variant, or a B.1.1.529, BA.1, BA.1.1, BA.2, BA.3, BA.4 and BA.5 lineage variant; or optionally the SARS-COV-2 is an alpha, beta, gamma, delta, epsilon, eta, iota, kappa, mu or zeta variant,
and optionally the coronavirus infection is a viral infection caused by COVID-19 or a variant, alternative clade or substrain thereof, or a Middle East respiratory syndrome virus (MERS-COV) infection,
and optionally the viral infection is caused by: a virus that causes a common cold, an influenza virus (optionally an influenza A, B or C), a hepatitis virus, a rous sarcoma virus (RSV), a Paramyxoviridae or measles virus, a Paramyxovirus or mumps virus, a Herpes simplex virus (HSV), a Cytomegalovirus (CMV), a Rubivirus or rubella virus, an Enterovirus, a viral meningitis, a rhinovirus, a human immunodeficiency virus (HIV), a varicella-zoster or chickenpox virus, an Orthopoxvirus or variola or smallpox virus, an Epstein-Barr virus (EBV), an Adenovirus, a Hantavirus, a Flaviviridae or Dengue virus, a Zika virus, or a chikungunya virus infection,

5. A pharmaceutical composition or a therapeutic combination of drugs of claim 1, or a product of manufacture of claim 2, or a kit of claim 3, for use in treating, preventing or ameliorating (optionally decreasing the symptoms of, or decreasing the mortality of) an infection caused by a virus, optionally a virus in the subfamily Orthocoronavirinae, or a virus in the family Coronaviridae, Arteriviridae, Roniviridae and/or Mesoniviridae, or a virus in the subfamily subfamily Letovirinae and Orthocoronavirinae; or a virus in the order Nidovirales, or an Alphacoronavirus, betacoronavirus, gammacoronavirus or deltacoronavirus, or a severe acute respiratory syndrome coronavirus 2 (SARS-COV-2) virus or variant thereof, and optionally the SARS-COV-2 is an omicron variant, or a B.1.1.529, BA.1, BA.1.1, BA.2, BA.3, BA.4 and BA.5 lineage variant; or optionally the SARS-COV-2 is an alpha, beta, gamma, delta, epsilon, eta, iota, kappa, mu or zeta variant,
and optionally the coronavirus infection is a viral infection caused by COVID-19 or a variant, alternative clade or substrain thereof, or a Middle East respiratory syndrome virus (MERS-COV) infection,
and optionally the viral infection is caused by: a virus that causes a common cold, an influenza virus (optionally an influenza A, B or C), a hepatitis virus, a rous sarcoma virus (RSV), a Paramyxoviridae or measles virus, a Paramyxovirus or mumps virus, a Herpes simplex virus (HSV), mumps virus (MuV), Human papillomavirus virus (HPV), a Cytomegalovirus (CMV), a Rubivirus or rubella virus, an Enterovirus, a viral meningitis, a vaccinia virus, a rhinovirus, a human immunodeficiency virus (HIV), a varicella-zoster or chickenpox virus, an Orthopoxvirus or variola or smallpox virus, an Epstein-Barr virus (EBV), an Adenovirus, a Hantavirus, a Flaviviridae or Dengue virus, ebola virus, a Zika virus, a pneumonia virus, a norovirus, a rotavirus, a virus of the genus Enterovirus or poliovirus, or a chikungunya virus infection.

6. A method for treating, preventing or ameliorating (optionally decreasing the symptoms of, or decreasing the mortality of) an infection caused by a virus, optionally a virus in the subfamily Orthocoronavirinae, or a virus in the family Coronaviridae, Arteriviridae, Roniviridae and/or Mesoniviridae, or a virus in the subfamily subfamily Letovirinae and Orthocoronavirinae; or a virus in the order Nidovirales, or an Alphacoronavirus, betacoronavirus, gammacoronavirus or deltacoronavirus, or a severe acute respiratory syndrome coronavirus 2 (SARS-COV-2) virus or variant thereof, and optionally the SARS-COV-2 is an omicron variant, or a B.1.1.529, BA.1, BA.1.1, BA.2, BA.3, BA.4 and BA.5 lineage variant; or optionally the SARS-COV-2 is an alpha, beta, gamma, delta, epsilon, eta, iota, kappa, mu or zeta variant,
and optionally the coronavirus infection is a viral infection caused by COVID-19 or a variant, alternative clade or substrain thereof, or a Middle East respiratory syndrome virus (MERS-COV) infection, and optionally the coronavirus infection is so-called “long COVID” or Post-COVID Conditions (PCC), or post-acute sequelae of SARS COV-2 infection (PASC), and optionally the viral infection is caused by: a virus that causes a common cold, an influenza virus (optionally an influenza A, B or C), a hepatitis virus, a rous sarcoma virus (RSV), a Paramyxoviridae or measles virus, a Paramyxovirus or mumps virus, a Herpes simplex virus (HSV), a Cytomegalovirus (CMV), a Rubivirus or rubella virus, an Enterovirus, a viral meningitis, a rhinovirus, a human immunodeficiency virus (HIV), a varicella-zoster or chickenpox virus, an Orthopoxvirus or variola or smallpox virus, an Epstein-Barr virus (EBV), an Adenovirus, a Hantavirus, a Flaviviridae or Dengue virus, a Zika virus, or a chikungunya virus infection,
the method comprising:
administering to an individual in need thereof of a pharmaceutically effective amount of a pharmaceutical composition or a therapeutic combination of drugs of claim 1, or a product of manufacture of claim 2, or a kit of claim 3.

7. The method of claim 6, wherein the avermectin class drug (optionally ivermectin) is dosaged or administered:
(a) (i) in or as a loading dosage comprising an avermectin class drug (optionally ivermectin) in a dosage of:
(1) about 300 μg/kg to 30 mg/kg (or 30 mg per 2.2 pounds (lb)) or about 18 mg to 1800 mg in a 60 kg (about 132 lb), or a dosage of 50 μg/kg, 75 μg/kg, 100 μg/kg or 500 μg/kg, or a dosage of between about 50 μg/kg and 500 μg/kg, or a loading dose of an avermectin class drug (optionally ivermectin) of between about 300 μg/kg per day to between 30 mg/kg to 60 mg/kg or between about 18 mg to about 1200 mg or 1600 mg to 1800 mg in a 60 kg (about 132 lb) person, or between about 300μg (mcg) to about 40 to 70 mg/kg, or a dosage of 60 to 120 mg to about 1600 to 1800 mg for an adult per day; or,
(2) between about 18 to 50 mg, or about 18 mg, 24 mg, 30 mg, 36 mg or 40 mg, or between about 50 mg to 100 mg, or 60 to 120 mg up to about 1600 to 1800 mg for an adult; and
wherein optionally the loading dosage is given once, or daily, or periodically, optionally every 2, 3, 4, 5, 6, 7, 8, 9, 10, 11, or 12 or more days,
(ii) after administration of the loading dosage of (i), administering a maintenance dosage of ivermectin of between about 20 mcg/kg (μ/kg) to 5000 mcg/kg (μ/kg) or between about 200 to 2000 mcg/kg (μ/kg) per dose, where 200 mcg/kg is equivalent to a 12 mg dosage in a 60 kg adult, and 2000 mcg/kg is equivalent to 120 mg per dose, or at about 50 μg/kg, 75 μg/kg or 100 μg/kg,
wherein optionally the maintenance dosage is administered 1, 2, 3, 4, 5, 6, 7, 8, 9, 10, 11, 12, 13 or 14 days, or every 3 weeks or every month or every two months or longer after the first loading dosage,
wherein optionally the maintenance dosage is administered every 1, 2, 3, 4, 5, 6, 7, 8, 9, 10, 11, 12, 13 or 14 days, every 3 weeks, or monthly, over the 4 to 8 weeks, 6 to 10 weeks, 8 to 12 weeks, 10 to 20 weeks, 15 to 30 weeks or 20 to 52 weeks, or more, after the initial or loading dose is given,
wherein optionally: an antibiotic or anti-viral is administered with the loading dosage of the avermectin class drug (optionally ivermectin); zinc (optionally zinc chelate, zinc salt or zinc sulfate) is administered with the loading dosage of the avermectin class drug (optionally ivermectin),
and optionally a drug combination, optionally formulated as one formulation (for example, as a tablet capsule) comprises: ivermectin, doxycycline and zinc chelate, or comprises: ivermectin 12 mg, doxycycline 100 mg and zinc chelate 25 mg, optionally administered once or twice daily; or
(b) a drug, a formulation or a therapeutic combination of drugs comprising an avermectin class drug (optionally ivermectin) at a dosage of:
(i) about 300 μg/kg to 30 mg/kg (or 30 mg per 2.2 pounds (lb)) or about 18 mg to 1800 mg in a 60 kg (about 132 lb), or a dosage of 50 μg/kg, 75 g/kg or 100 μg/kg, or at a loading dose of ivermectin of between about 30 μg/kg to 60 mg/kg or between about 18 mg to about 1200 mg or 1600 mg to 1800 mg in a 60 kg (about 132 lb) person, or between about 40 to 70 mg/kg, or a dosage of 60 to 120 mg to about 1600 to 1800 mg, or is dosage at 50 μg/kg, 75 μg/kg, 100 μg/kg or 500 μg/kg, or between about 50 μg/kg and 500 μg/kg, for an adult, or
(ii) between about 18 to 50 mg, or about 18 mg, 24 mg, 30 mg, 36 mg or 40 mg, or between about 50 mg to 100 mg, or 60 to 120 mg to about 1600 to 1800 mg for daily an adult.

8. The method of claim 6 or claim 7, wherein an additional drug or drugs is dosaged or administered with the metformin and/or the 3C-like (3CLpro) protease or ensitrelvir, wherein the additional drug or drugs comprises:
(a) combination of an avermectin class drug (optionally ivermectin) (optionally dosaged at between about 30 to 80 mg per day, or between about 36 to 60 mg per day), clofazimine (optionally dosaged at about 100 mg or 150 mg per day, or between about 50 mg and 200 mg per day) and zinc (optionally a zinc sulphate, acetate, gluconate or picolinate, or zinc oxide nanoparticles, optionally at a dosage of between about 1 mg to 250 mg, or about 50 mg per day), and optionally also including colchicine;
(b) a combination of clofazimine (optionally dosaged at about 100 mg or 150 mg per day, or between about 50 mg and 200 mg per day), fluvoxamine, and zinc (optionally a zinc sulphate, acetate, gluconate or picolinate, or zinc oxide nanoparticles, optionally at a dosage of between about 1 mg to 250 mg, or about 50 mg per day), and optionally also including colchicine; or
(c) a combination of an avermectin class drug (optionally ivermectin) (optionally dosaged at between about 10 to 100 mg per day or between about 12 to 80 mg per day, or between about 36 to 60 mg per day), clofazimine (optionally dosaged at about 100 mg or 150 mg per day, or between about 50 mg and 200 mg per day), fluvoxamine and at least one vitamin, wherein optionally the at least one vitamin comprises: vitamin B3 (or pyridine-3-carboxylic acid, niacin or nicotinic acid, or vitamin B3 or niacin administered as a slow release form (or NIASPAN FCT™), vitamin D (optionally D2, or ergocalciferol), or Vitamin D3 or cholecalciferol, optionally administered at about 1000 to 4000 ugm/day; vitamin B12, vitamin B6 (or pyridoxine); vitamin K; vitamin A; vitamin E; and/or, vitamin C (optionally administered at 500 mg bid), and optionally further comprising zinc (optionally a zinc sulphate, acetate, gluconate or picolinate, or zinc oxide nanoparticles, optionally at a dosage of between about 1 mg to 250 mg), and optionally also including colchicine; hydrocortisone or cortisol (optionally CORTEF™, SOLUCORTEF™), optionally hydrocortisone sodium succinate or hydrocortisone acetate or dexamethasome (optionally DEXTENZA™, OZURDEX™, NEOFORDEX™).

9. The method of any one of claims 6 to 8, wherein the pharmaceutical composition or a therapeutic combination of drugs of claim 1, or the product of manufacture of claim 2, or the kit of claim 3, is formulated as, is administered as, or comprises:
a pharmaceutical formulation, or a dressing or a dermal patch, or an implant,
and optionally the pharmaceutical composition or a therapeutic combination is formulated as: gel, a liquid, an aerosol, a spray, a mist, a lotion, a cream, a powder, a tablet, a capsule, a pill, a geltab, an enema, a suppository, a transdermal or intraoral patch, or an injectable formulation, a subcutaneous, an intramuscular (IM) or intravenous (IV) formulation, to make a pharmaceutical formulation.

10. The method of any one of claims 6 to 9, wherein:
(a) the antibiotic, or macrolide drug, optionally doxycycline or azithromycin, is administered by starting with a loading dose, optionally an oral, IV or IM dosage of between about 400 mg to 500 mg and 1 g, or at about 500 mg, optionally with follow up administrations daily every 4 to 10 days, or every 1, 2, 3, 4, 5, 6, 7, 8, 9, 10 days or up to 20 or more days, at a lower dosage of between about 100 gm to 300 mg, or about 250 mg, total daily dosage, optionally continuing for between about one week to one month;
(b) the azithromycin is administered (optionally, ZITHROMAX™, or AZITHROCIN™) at a 500 mg bid loading dose on day one, then 500 mg in the morning (MANE) for days two, three and four, then azithromycin ceased and replaced by doxycycline (optionally, DORYX™, DOXYHEXA™, DOXYLIN™) 100 mg bid for the remainder of the treatment (ten or eleven days); or
(c) the azithromycin is first administered (optionally, ZITHROMAX™, or AZITHROCIN™) at a 500 mg bid loading dose on day one, then 500 mg in the morning (MANE) for days two, three and four, then azithromycin ceased, and doxycycline 100 mg bid (or between about 25 to 500 mg bid) (optionally, DORYX™, DOXYHEXA™, DOXYLIN™) every day for the full duration of the treatment (ten or eleven days, or more).

11. The method of any one of claims 6 to 10, wherein the administration of the pharmaceutical composition or a therapeutic combination of drugs of claim 1, or the product of manufacture of claim 2, or the kit of claim 3, or treatment, lasts between about 10 days and 3 weeks, or 11 days and 2 weeks, or for about 10, 11, 12, 13 or 14 days, or between about one month and one year.

12. The method of any one of claims 6 to 10, wherein zinc, optionally zinc sulfate or zinc chelate, is administered at a dosage of 100 mg MANE, or between about 50 mg to 150 mg, every day of the treatment or administration.

13. A transdermal device comprising or manufactured to have: a plurality of or multiple drug containing or drug storage compartments (or cells or wells), each compartment sealed or having contiguous walls such that when a drug formulation (optionally a liquid, liposome, gel or hydrogel drug formulation) is placed inside one of the plurality of or multiple drug containing or drug storage compartments or cells or wells the liquid or gel drug formulation is contained in the compartment, and the compartment has one side open to a removable (and optionally a flexible) release liner which can be removed by a user, and on the same side of the transdermal device as the open side of (or side covered by the release liner) the compartment or cell or well to the flexible and removable release liner is affixed an adhesive (optionally comprising an acrylate polymer or a pressure-sensitive adhesive (PSA)) to affix the transdermal device to a skin surface, wherein the adhesive is covered until removal of the removable release liner.

14. The transdermal device of claim 13, wherein the transdermal device comprises between 2 and 10 separate, or individual, drug containing or drug storage cells or compartments, and optionally each of the multiple compartments (or cells, or wells) has a storage capacity of between about 5 μl and 1000 μl (or one ml), or between about 20 μl and 500 μl, or between about 50 μl and 5 ml.

15. The transdermal device of claim 13, further comprising a pressure cuff, wherein the transdermal device is set, affixed, glued or sewn onto a cuff or band designed or manufactured to be worn around an arm (or an upper arm or lower arm), or leg, and the cuff or band is designed or manufactured to allow a user to tighten the cuff or band around the arm or leg, optionally by use of a hook and loop fastener (a lineal fabric strip with tiny hooks that can mate with another fabric strip with smaller loops, attaching temporarily, until pulled apart) (or VELCRO™), or the cuff or band is designed or manufactured to tighten around the arm or leg by inflation (as is a sphygmo-manometer, or a blood pressure cuff), wherein the cuff or band is wrapped around an arm, or upper arm, and inflated, and optionally cuff or band is manufactured or designed as described in U.S. Pat. No. 10,398,325.

16. The transdermal device of claims 13 or 14, wherein the transdermal device comprises 3 separate, or individual, drug containing or drug storage cells or compartments.

17. The transdermal device of claim 16, wherein a first compartment comprises an avermectin class drug (optionally ivermectin), a second compartment comprises an antibiotic (optionally doxycycline or azithromycin) and a third compartment comprises zinc (optionally a zinc chelate such as zinc sulphate, acetate, gluconate or picolinate, or zinc oxide nanoparticles).

18. The transdermal device of claim 16 or 17, wherein a first compartment comprises ivermectin, a second compartment comprises doxycycline and a third compartment comprises zinc chelate.

19. The transdermal device of claim 17 or 18, wherein the first compartment comprises 48 mg of ivermectin, the second compartment comprises 300 mg of doxycycline and the third compartment comprises 50 mg of zinc chelate.

20. A method for treating, preventing or ameliorating (optionally decreasing the symptoms of, or decreasing the mortality of) a coronavirus infection caused by COVID-19, or a variant, alternative clade or substrain thereof, the method comprising administering to an individual in need thereof the transdermal device of any one of claims 13 to 19.

21. Use of the transdermal device of any one of claims 13 to 19 for the manufacture of a medicament for preventing or ameliorating (optionally decreasing the symptoms of, or decreasing the mortality of) a coronavirus infection caused by COVID-19, or a variant, alternative clade or substrain thereof.

22. The transdermal device of any one of claims 13 to 19 for use in treating, preventing or ameliorating (optionally decreasing the symptoms of, or decreasing the mortality of) a coronavirus infection caused by COVID-19, or a variant, alternative clade or substrain thereof.
